(19) United States
(12) Patent Application Publication
(10) Pub. No.: US 2026/0325747 A1
(43) Pub. Date: Oct. 22, 2026

(54) PRESSURE RELEASE ASSEMBLY FOR FLUID INJECTOR TUBE SET

(71) Applicant: Bayer Healthcare LLC, Whippany, NJ (US)

(72) Inventors: Kevin COWAN, Allison Park, PA (US); John HAURY, Sewickley, PA (US); Michael SPOHN, Fenelton, PA (US); James DEDIG, Pittsburgh, PA (US); Corey SAVANNAH, Butler, PA (US)

(21) Appl. No.: 19/119,232

(22) PCT Filed: Oct. 17, 2023
(86) PCT No.: PCT/US2023/035311

§ 371 Date: Apr. 8, 2025

Related U.S. Application Data
(60) Provisional application No. 63/417,097, filed on Oct. 18, 2022.

(51) Int. Cl. A61M 5/145; A61M 5/00; A61M 5/14; A61M 5/31

(52) U.S. Cl. CPC A61M 5/1452; A61M 5/007; A61M 2005/1402; A61M 2005/3128

(57) ABSTRACT

A pressure release assembly for a tubing set may include an engagement sleeve including an inner surface defining a cavity, the inner surface including a connection arrangement configured to connect the engagement sleeve to a tubing set, and a pin member configured to be received in the cavity of the engagement sleeve. The pin member may be movable between a first position in which the pin member is removed from a seal of the tubing set and a second position in which the pin member is inserted into the seal of the tubing set to allow air to be purged from the tubing set.

### CROSS REFERENCE TO RELATED APPLICATIONS

This application claims the benefit of U.S. Provisional Patent Application No. 63/417,097, filed on 18 Oct. 2022, the disclosure of which is incorporated in its entirety by reference.

### BACKGROUND

### Field of the Invention

The present disclosure is related generally to features associated with fluid path elements for powered medical fluid injection systems. The described features provide a pressure release assembly for fluid path elements of fluid injector tubing sets that allow for release of pressure during a priming operation through a swabable valve of the tubing set.

### Description of Related Art

Syringe injection systems are among the medical devices used in medical imaging procedures for many years. Many such syringes are operated manually by advancing a piston into operative connection with an internal plunger to pressurize a fluid within the syringe. To reduce the number of disposable components during a sequence of fluid injections, systems may incorporate a multi-patient portion, such as a multi-patient pump system and multi-patient fluid path elements, that may be used over a series of fluid injection procedures; and a single-patient fluid path element, including associated check valves, that are used for only a single injection procedure and then disposed of and replaced with a new, sterile single-patient fluid path element for a subsequent fluid injection procedure.

A number of syringe-based or peristaltic pump-based powered injectors have been developed for administering a fluid, such as a contrast media or saline, in medical imaging procedures such as cardiovascular angiography (CV), computed tomography (CT), positron emission tomography (PET), and nuclear magnetic resonance (NMR)/magnetic resonance imaging (MRI). Some of these powered injectors may include options for use in a multi-patient configuration utilizing multi-use components and single-use components. One example of such a system is the Bayer MEDRAD® Centargo CT Fluid Injection System, including multi-patient and single-patient components as described in U.S. Pat. Nos. 10,507,319 and 10,549,084, the disclosures of which are incorporated by reference herein.

Since the various multi-patient elements are used over several fluid injection procedures, steps must be taken to ensure that these elements are sterile and not exposed to microbial contaminants from previous patients or the environment that may be harmful to later patients. For example, many systems may include a swabable valve that may be disinfected by a technician wiping the valve with an alcohol-soaked wipe in between injection procedures.

Conventional systems having swabable valves include standard Luer-type connectors between the multi-patient and single-patient components which are fitted with off-the-shelf swabable valves on the female Luer component and may include a threaded connection mechanism. Examples of a threaded connector for use in a multi-patient configuration are described in U.S. Pat. No. 8,540,698, the disclosure of which is incorporated by reference herein. Various embodiments of swabable valves may include an elastomeric cap portion with a slit that may engage a male luer to provide fluid communication between the fluid path component with the swabable valve and the fluid path component with the male luer connector. The elastomeric cap portion with the slit prevent fluid flow through the swabable valve when disconnected from the male Luer component, as the slit in the elastomeric material effectively seals to create a fluid-tight barrier. Prior to connection, the exterior surface of the swabable valve may be wiped or swabbed with a disinfecting cloth such as an alcohol wipe to remove any microbial contaminants from the exterior surface and prevent contamination of the fluid path interior.

In certain multi-patient injection procedures, a tubing path sealed with a swabable valve may need to be purged of air prior to connection. Purging of air may be performed by flowing pressurized fluid through the tubing path to discharge the air from the tubing path. However, conventional swabable valves may remain sealed under pressures of up to 100 psi or more before the seal inverts and allows air and fluid to flow therethrough. In certain situations, this may result in release of a volume of fluid in addition to the air during the purging operation, resulting in fluid contamination of medical surfaces and injector components and waste of medical fluid. New fluid path component configurations that allow air to be readily purged under low fluid pressures, for example without escape of medical fluid, are required and that can allow swabbing disinfection of the fluid path valve are needed.

### BRIEF SUMMARY

The present disclosure provides fluid path elements, fluid injector systems, and methods of using a swabable valve assembly including a pressure release assembly for fluid path elements of fluid injector tubing sets that allow for release of volumes of air during a priming operation through a swabable valve of the tubing set. The pressure release assembly allows release of air through the outlet of the swabable valve attached to a tubing set during a priming or purging operation without significant release of the fluid as the fluid is pressurized to prime/purge the tubing set. The pressure release assembly may include a cap removably attachable to a swabable valve assembly connected to a distal end of a tubing set. The cap may be threadably attached to the swabable valve assembly or attached by other conventional means. The cap may be removed from the swabable valve assembly after the priming or purging operation so that the swabable valve may be sterilized and/or connected to another fluid path component, such as a spike for fluidly connecting the tubing set to a bulk fluid container. The cap may include a distal opening for insertion of a pressure release assembly therethrough. In certain embodiments, the pressure release assembly may include a throughbore running from a proximal end to a distal opening to allow pressurized air to travel out of the tubing set. The proximal end may be configured for insertion into or through the swabable valve, for example through the slit in the elastomeric cap portion, to provide fluid communication between the interior of the tubing set to the exterior of the pressure release assembly through the distal opening of the pressure release assembly when inserted through the swabable valve. The pressure release assembly may be movable between a first, non-insertion position where the proximal end of the pressure release assembly is not inserted through the swabable valve and no fluid communication exists between the interior of the tubing set and the distal opening of the pressure release assembly, and a second, insertion position where the proximal end of the pressure release assembly is inserted through the swabable valve. In the second, insertion position, the pressure release assembly may allow purging or priming of air from the tubing set through the swabable valve. In certain embodiments, the pressure release assembly may be manually movable between the first and second positions. In certain embodiments, the pressure release assembly may be moved by the fluid injector between the first and second positions. In certain embodiments, the pressure release assembly may be reversibly movable between the first and second positions. In other embodiments, the pressure release assembly may be locked in the second position once moved to the second position.

In certain embodiments, at least a portion of the tubing elements including a portion of the tubing set, the swabable valve, and the pressure release assembly may be configured for insertion into a sensor, for example an air and/or fluid sensor associated with the fluid injector and in communication with a controller of the fluid injector system. In certain embodiments, the sensor may detect, for example, an interface between the air and liquid in the tubing set and control the purging or priming procedure so that essentially all of the air is removed from the tubing set. In certain embodiments, the sensor may send a signal to the controller to stop a purging or priming procedure when the sensor detects the interface between the air and liquid at a location just proximal to the elastomeric cap portion of the swabable valve. In this manner, essentially all of the air within the tubing set may be removed from the tubing set during the purging or priming procedure without any liquid being spilled or ejected through the swabable valve and pressure release assembly, thereby preventing any spilling of medical liquid or fouling of the fluid injector components or components in the injection suite. In other embodiments, for example, where the pressure release assembly is configured to hold small volumes of liquid, the sensor may detect when the interface between the air and liquid passes through the swabable valve into the pressure release assembly.

In various embodiments, once the tubing set is purged or primed of the majority of or all air, the pressure release assembly may be removed from the swabable valve and the swabable valve and tubing set may be connected to another fluid path component, such as a spike for spiking a bulk fluid source, for example, when additional fluid is required to be loaded into the syringe for one or more additional injection procedures. According to this embodiment, any remaining small amounts of air may be purged into the bulk fluid source after attaching the spike, for example by pressurizing a syringe or activating a peristaltic pump and the associated fluid path components.

According to one non-limiting embodiment or aspect of the present disclosure, a pressure release assembly may include an engagement sleeve comprising an inner surface defining a cavity, the inner surface comprising a connection arrangement configured to connect the engagement sleeve to a tubing set and/or an associated swabable valve; and a pin member configured to be received in the cavity of the engagement sleeve, in which the pin member is movable between a first position in which the pin member is removed from a seal of the tubing set and/or swabable valve and a second position in which the pin member is inserted into the seal of the tubing set and/or swabable valve to allow a volume of air to be purged from the tubing set through the pressure release assembly.

According to one non-limiting embodiment or aspect of the present disclosure, the connection arrangement of the engagement sleeve comprises a threaded surface to threadedly connect the engagement sleeve to the tubing set, for example, to a corresponding treaded surface on a swabable valve. The pin member may comprise a flange provided on a distal end of the pin member. The pin member defines a throughbore that extends through the pin member, and wherein, when the pin member is held in the second position, the throughbore establishes a fluid communication with the interior of the tubing set to allow the air to be purged from the tubing set. The pin member may define at least one detent, and wherein, when the pin member is held in the first position, a portion of the engagement sleeve engages with the at least one detent to hold the pin member in the first position. The portion of the engagement sleeve that engages with the at least one detent comprises at least one flexible engagement member for flexibly engaging the at least one detent. In certain embodiments, the pin member may define a first detent and a second detent, defined at a distance from the first detent, and wherein, when the pin member is held in the first position, a portion of the engagement sleeve engages with the first detent to holds the pin member in the first position, and wherein, when the pin member is held in the second position, the portion of the engagement sleeve engages with the second detent to hold the pin member in the second position. In certain embodiments, the pin member may define a throughbore that extends through at least a portion of the pin member, for example along at least a portion of a longitudinal axis of the pin member, and wherein a filter is positioned in the throughbore to permit air to move through the throughbore while preventing fluid from moving through the throughbore past the filter. In certain embodiments, an outer surface of the engagement sleeve may comprise at least one gripping element. In certain embodiments, an outer surface of the pin member may comprise a corresponding connection arrangement that is configured to engage with the connection arrangement of the engagement sleeve to move the pin member between the first position and the second position. In certain embodiments, the engagement sleeve comprises a flexible arm member configured to engage at least one detent defined on the pin member. In certain embodiments, the pin member comprises a ring slidably received on an outer surface of the pin member, and wherein the ring is pushed from a first indication position to a second indication position by a portion of the engagement sleeve when the pin member is moved from the first position to the second position to indicate the pin member has broken the seal of the tubing set. In certain embodiments, the engagement sleeve defines a vent configured to permit air to pass therethrough once the pin member has been moved to the second position.

In other embodiments, the outer surface of the pin member may comprise at least one longitudinal groove. The at least one longitudinal groove may provide fluid communication with the interior of the tubing set, for example through the swabable valve component of the pressure release assembly, wherein in the second position the at least one longitudinal groove prevents a fluid-tight seal with the swabable valve component.

According to one non-limiting embodiment or aspect of the present disclosure, a fluid injector, comprises a housing; at least one syringe removably engaged with the housing; a tubing set configured to fluidly connect the at least one syringe at a proximal end and having a swabable valve at a distal end, and at least one fluid reservoir that holds fluid to fill the at least one syringe; and a pressure release assembly configured to connect to the distal end of tubing set to purge air from the tubing set, the pressure release assembly comprising: an engagement sleeve comprising an inner surface defining a cavity, the inner surface comprising a connection arrangement configured to removably connect the engagement sleeve to the tubing set; and a pin member configured to be received in the cavity of the engagement sleeve, wherein the pin member is movable between a first position in which the pin member is removed from a seal of the swabable valve at the distal end of the tubing set and a second position in which the pin member is inserted into the seal of the swabable valve at the distal end of the tubing set to allow air to be purged from the tubing set.

According to one non-limiting embodiment or aspect of the fluid injector of the present disclosure, the fluid injector may further comprise a cassette for holding the tubing set wherein the cassette includes a body defining at least one feature for holding at least a portion of the tubing set having a pressure release assembly as described herein; and a connecting member for removably connecting the body of the cassette to the fluid injector, the connecting member operatively connected to the body. In certain embodiments, the pressure release assembly is configured to operatively engage the release assembly cassette to move the pressure release assembly from the first position to the second position. The connection arrangement of the engagement sleeve comprises a threaded surface to threadedly connect the engagement sleeve to the tubing set. The pin member comprises a flange provided on an end of the pin member. The pin member defines a throughbore that extends longitudinally through the pin member from a proximal end to a distal end, and wherein, when the pin member is held in the second position, the throughbore establishes a fluid communication with the tubing set through an opening at the distal end of the throughbore to allow the air to be purged from the tubing set. The pin member defines at least one detent, and wherein, when the pin member is held in at least one of the first position and the second position, a portion of the engagement sleeve engages with the at least one detent to hold the pin member in at least one of the first position and the second position. The portion of the engagement sleeve that engages with the at least one detent may comprise at least one flexible engagement member. In certain embodiments, the pin member may define a first detent and a second detent, defined at a distance from the first detent, and wherein, when the pin member is held in the first position, a portion of the engagement sleeve engages with the first detent to holds the pin member in the first position, and wherein, when the pin member is held in the second position, the portion of the engagement sleeve engages with the second detent to hold the pin member in the second position. In certain embodiments, the pin member may further comprise a filter positioned in the throughbore to permit air to move through the throughbore while preventing fluid from moving through the throughbore. An outer surface of the engagement sleeve comprises at least one gripping element. The outer surface of the tubing set and/or the pressure release assembly is configured to releasably engaged with the cassette to hold the tubing set and/or the pressure release assembly in operative connection with the cassette. An outer surface of the pin member comprises a corresponding connection arrangement that is configured to engage with the connection arrangement of the cassette to move the pin member between the first position and the second position. The pin member comprises a ring slidably received on an outer surface of the pin member, and wherein the ring is pushed from a first indication position to a second indication position by a portion of the engagement sleeve when the pin member is moved from the first position to the second position to indicate the pin member has broken the seal of the tubing set. The engagement sleeve defines a vent configured to permit air to pass therethrough once the pin member has been moved to the second position. An activation member is provided on the cassette to engage the pin member of the pressure release assembly to move the pin member from the first position to the second position. In certain embodiments, the activation member may engage the pin member when the tubing set and/or pressure release assembly is manually inserted into the cassette to move the pressure release assembly and associated pin from the first position to the second position. In other embodiments, the activation member may be in communication with a controller associated with the fluid injector, wherein the activation member activates the pressure release assembly and moves the pin from the first position to the second position, in response to a signal from the controller. The activation member may comprise a solenoid valve or other electromechanical component configured to move the pin of the pressure release assembly.

According to one non-limiting embodiment or aspect of the present disclosure, a method of purging air from a tubing set may include connecting a pressure release assembly to a distal end of the tubing set, for example to a swabable valve at the distal end of the tubing set, the pressure release assembly comprising an engagement sleeve comprising an inner surface defining a cavity, the inner surface comprising a connection arrangement configured to connect the engagement sleeve to the tubing set, and a pin member configured to be received in the cavity of the engagement sleeve; moving the pin member from a first position to a second position to break a seal at the distal end of the tubing set and insert a proximal end of the pin member into the tubing set; and purging air from the tubing set through the pressure release assembly. In certain embodiments, the air may be purged through one or more grooves on an outer surface of the pin and through a vent defined in the engagement sleeve of the pressure release assembly. In other embodiments, the pin member defines a throughbore extending from a proximal end of the pin to the distal end of the pin or to a vent through an outer surface of the pin between the proximal end and the distal end of the pin, that establishes fluid communication with the tubing set when the pin member is in the second position such that the air is purged through the throughbore of the pin member.

The various features of present disclosure are further defined by the following clauses:
Clause 1: A pressure release assembly for a tubing set, the pressure release assembly comprising: an engagement sleeve comprising an inner surface defining a cavity, the inner surface comprising a connection arrangement configured to connect the engagement sleeve to the tubing set and/or an associated swabable valve; and a pin member configured to be received in the cavity of the engagement sleeve, wherein the pin member is movable between a first position in which the pin member is removed from a seal of the tubing set and/or the associated swabable valve and a second position in which the pin member is inserted into the seal of the tubing set and/or the associated swabable valve to allow a volume of air to be purged from the tubing set through the pressure release assembly.
Clause 2: The pressure release assembly of Clause 1, wherein the connection arrangement of the engagement sleeve comprises a threaded surface to threadedly connect the engagement sleeve to the tubing set.
Clause 3: The pressure release assembly of Clause 1 or Clause 2, wherein the pin member comprises a flange provided on a distal end of the pin member.
Clause 4: The pressure release assembly of any of Clauses 1-3, wherein the pin member defines a throughbore that extends through the pin member, and wherein, when the pin member is held in the second position, the throughbore establishes a fluid communication with the interior of the tubing set to allow the air to be purged from the tubing set.
Clause 5: The pressure release assembly of any of Clauses 1-4, wherein the pin member defines at least one detent, and wherein, when the pin member is held in the first position, a portion of the engagement sleeve engages with the at least one detent to hold the pin member in the first position.
Clause 6: The pressure release assembly of Clause 5, wherein the portion of the engagement sleeve that engages with the at least one detent comprises at least one flexible engagement member for flexibly engaging the at least one detent.
Clause 7: The pressure release assembly of any of Clauses 1-6, wherein the pin member defines a first detent and a second detent, defined at a distance from the first detent, and wherein, when the pin member is held in the first position, a portion of the engagement sleeve engages with the first detent to hold the pin member in the first position, and wherein, when the pin member is held in the second position, the portion of the engagement sleeve engages with the second detent to hold the pin member in the second position.
Clause 8: The pressure release assembly of any of Clauses 1-7, wherein the pin member defines a throughbore that extends through at least a portion of the pin member, and wherein a filter is positioned in the throughbore to permit air to move through the throughbore while preventing fluid from moving through the throughbore past the filter.
Clause 9: The pressure release assembly of any of Clauses 1-8, wherein an outer surface of the engagement sleeve comprises at least one gripping element.
Clause 10: The pressure release assembly of any of Clauses 1-9, wherein an outer surface of the pin member comprises a corresponding connection arrangement that is configured to engage with the connection arrangement of the engagement sleeve to move the pin member between the first position and the second position.
Clause 11: The pressure release assembly of any of Clauses 1-10, wherein the engagement sleeve comprises a flexible arm member configured to engage at least one detent defined on the pin member.
Clause 12: The pressure release assembly of any of Clauses 1-11, wherein the pin member comprises a ring slidably received on an outer surface of the pin member, and wherein the ring is pushed from a first indication position to a second indication position by a portion of the engagement sleeve when the pin member is moved from the first position to the second position to indicate the pin member has broken the seal of the tubing set.
Clause 13: The pressure release assembly of any of Clauses 1-12, wherein the engagement sleeve defines a vent configured to permit air to pass therethrough once the pin member has been moved to the second position.
Clause 14: A fluid injector, comprising: a housing; at least one syringe removably engaged with the housing; a tubing set configured to fluidly connect the at least one syringe at a proximal end and having a swabable valve at a distal end, and at least one fluid reservoir that holds fluid to fill the at least one syringe; and a pressure release assembly configured to connect to the distal end of the tubing set to purge air from the tubing set, the pressure release assembly comprising: an engagement sleeve comprising an inner surface defining a cavity, the inner surface comprising a connection arrangement configured to removably connect the engagement sleeve to the tubing set; and a pin member configured to be received in the cavity of the engagement sleeve, wherein the pin member is movable between a first position in which the pin member is removed from a seal of the swabable valve at the distal end of the tubing set and a second position in which the pin member is inserted into the seal of the swabable valve at the distal end of the tubing set to allow air to be purged from the tubing set.
Clause 15: The fluid injector of Clause 14, further comprising a cassette for holding the tubing set, the cassette comprising: a body defining at least one feature for holding at least a portion of the tubing set; and a connecting member for removably connecting the body of the cassette to the fluid injector, the connecting member operatively connected to the body.
Clause 16: The fluid injector of Clause 14 or Clause 15, wherein the connection arrangement of the engagement sleeve comprises a threaded surface to threadedly connect the engagement sleeve to the tubing set.
Clause 17: The fluid injector of any of Clauses 14-16, wherein the pin member comprises a flange provided on an end of the pin member.
Clause 18: The fluid injector of any of Clauses 14-17, wherein the pin member defines a throughbore that extends longitudinally through the pin member from a proximal end to a distal end, and wherein, when the pin member is held in the second position, the throughbore establishes a fluid communication with the tubing set through an opening at the distal end of the throughbore to allow the air to be purged from the tubing set.
Clause 19: The fluid injector of any of Clauses 14-18, wherein the pin member defines at least one detent, and wherein, when the pin member is held in at least one of the first position and the second position, a portion of the engagement sleeve engages with the at least one detent to hold the pin member in at least one of the first position and the second position.
Clause 20: The fluid injector of Clause 19, wherein the portion of the engagement sleeve that engages with the at least one detent comprises at least one flexible engagement member.
Clause 21: The fluid injector of any of Clauses 14-20, wherein the pin member defines a first detent and a second detent, defined at a distance from the first detent, and wherein, when the pin member is held in the first position, a portion of the engagement sleeve engages with the first detent to hold the pin member in the first position, and wherein, when the pin member is held in the second position, the portion of the engagement sleeve engages with the second detent to hold the pin member in the second position.
Clause 22: The fluid injector of any of Clauses 14-21, wherein the pin member defines a throughbore that extends through the pin member, and wherein a filter is positioned in the throughbore to permit air to move through the throughbore while preventing fluid from moving through the throughbore.
Clause 23: The fluid injector of any of Clauses 14-22, wherein an outer surface of the engagement sleeve comprises at least one gripping element.
Clause 24: The fluid injector of any of Clauses 14-23, wherein an outer surface of the pin member comprises a corresponding connection arrangement that is configured to engage with the connection arrangement of the cassette to move the pin member between the first position and the second position.
Clause 25: The fluid injector of any of Clauses 14-24, wherein the pin member comprises a ring slidably received on an outer surface of the pin member, and wherein the ring is pushed from a first indication position to a second indication position by a portion of the engagement sleeve when the pin member is moved from the first position to the second position to indicate the pin member has broken the seal of the tubing set.
Clause 26: The fluid injector of any of Clauses 14-25, wherein the engagement sleeve defines a vent configured to permit air to pass therethrough once the pin member has been moved to the second position.
Clause 27: The fluid injector of any of Clauses 14-26, further comprising an activation member provided on the cassette to engage the pin member of the pressure release assembly to move the pin member from the first position to the second position.
Clause 28: The fluid injector of Clause 27, wherein the activation member comprises a solenoid valve or other electromechanical component configured to move the pin member of the pressure release assembly.
Clause 29: A method of purging air from a tubing set, the method comprising: connecting a pressure release assembly to a distal end of the tubing set, the pressure release assembly comprising an engagement sleeve comprising an inner surface defining a cavity, the inner surface comprising a connection arrangement configured to connect the engagement sleeve to the tubing set, and a pin member configured to be received in the cavity of the engagement sleeve; moving the pin member from a first position to a second position to break a seal at the distal end of the tubing set and insert a proximal end of the pin member into the tubing set; and purging air from the tubing set through the pressure release assembly.
Clause 30: The method of Clause 29, wherein the air is purged through one or more grooves on an outer surface of the pin member and through a vent defined in the engagement sleeve of the pressure release assembly.

Clause 31: The method of Clause 29, wherein the pin member defines a throughbore extending from a proximal end of the pin member to the distal end of pin member or to a vent through an outer surface of the pin member between the proximal end and the distal end of the pin member that establishes fluid communication with the tubing set when the pin member is in the second position such that the air is purged through the throughbore of the pin member.

### BRIEF DESCRIPTION OF THE DRAWINGS

The novel features herein are set forth with particularity in the appended claims. Such features, however, both as to organization and methods of operation, may be better understood by reference to the following description, in conjunction with the accompanying drawings.

FIG. 1 is a perspective view of a pressure release assembly and swabable valve in a first, non-insertion position according to one non-limiting embodiment of the present application;

FIG. 2 is a cross-sectional view of the pressure release assembly and swabable valve of FIG. 1;

FIG. 3 is a perspective view of the pressure release assembly and swabable valve of FIG. 1 provided in a second, insertion position;

FIG. 4 is a cross-sectional view of the pressure release assembly and swabable valve of FIG. 3;

FIG. 5 is a perspective view and front view of a pin member according to one non-limiting embodiment of the present disclosure;

FIG. 6 is a perspective view and front view of a pin member according to another non-limiting embodiment of the present disclosure;

FIG. 7 is a cross-sectional view of a pressure release assembly with the pin member of FIG. 5 shown in a first, non-insertion position;

FIG. 8 is a cross-sectional view of a pressure release assembly with the pin member of FIG. 6 shown in a first, non-insertion position;

FIG. 9 is a cross-sectional view of a pressure release assembly with the pin member of FIG. 5 shown in a second, insertion position;

FIG. 10 is a cross-sectional view of a pressure release assembly with the pin member of FIG. 6 shown in a second, insertion position;

FIG. 11 is a cross-sectional view of a pressure release assembly with a pin member having a side vent according to another non-limiting embodiment of the present disclosure shown in a first, non-insertion position;

FIG. 12 is a cross-sectional view of the pressure release assembly of FIG. 11 shown in a second, insertion position;

FIG. 13 is a perspective view of a pressure release assembly and swabable valve provided in a first, non-insertion position according to one non-limiting embodiment;

FIG. 14 is a cross-sectional view of the pressure release assembly and swabable valve of FIG. 13;

FIG. 15 is a perspective view of the pressure release assembly and swabable valve of FIG. 13 provided in a second, insertion position;

FIG. 16 is a cross-sectional view of the pressure release assembly and swabable valve of FIG. 15;

FIG. 17 is a perspective view of a pressure release assembly and swabable valve provided in a first, non-insertion position according to one non-limiting embodiment;

FIG. 18 is a cross-sectional view of the pressure release assembly and swabable valve of FIG. 17;

FIG. 19 is a cross-sectional view of the pressure release assembly and swabable valve of FIG. 17 provided in a second, insertion position;

FIG. 20 is a side view of a pressure release assembly and swabable valve of FIG. 17 provided in a first, non-insertion position disengaged with a cassette of a fluid injector according to one non-limiting embodiment of the present application;

FIG. 21 is a side view of cassette of the fluid injector engaged with the pressure release assembly and swabable valve of FIG. 20 provided in a second, insertion position in the injector module;

FIG. 22 is a cross-sectional view of a pressure release assembly and swabable valve provided in a first, non-insertion position according to one non-limiting embodiment;

FIG. 23 is a cross-sectional view of the pressure release assembly and swabable valve of FIG. 22 provided in a second, insertion position;

FIG. 24 is a cross-sectional view of a pressure release assembly and swabable valve provided in a first, non-insertion position according to one non-limiting embodiment;

FIG. 25 is a cross-sectional view of the pressure release assembly and swabable valve of FIG. 24 provided in a second, insertion position;

FIG. 26 is a cross-sectional view of the pressure assembly and swabable valve of FIG. 24 shown with a prime tube attached; and

FIG. 27 is a side view of a pressure release assembly and swabable valve provided in a second, insertion position engaged with a cassette of a fluid injector according to one non-limiting embodiment of the present application.

### DESCRIPTION OF THE DISCLOSURE

In the following detailed description, reference is made to the accompanying drawings, which form a part hereof. The illustrative features shown and described in the detailed description, drawings, and claims are not meant to be limiting. Other features may be utilized, and other changes may be made, without departing from the scope of the subject matter presented here.

Before explaining the various aspects of the pressure release assembly and various features thereof in detail, it should be noted that the various aspects disclosed herein are not limited in their application or use to the details of construction and arrangement of parts illustrated in the accompanying drawings and description. Rather, the disclosed devices may be positioned or incorporated in other devices, variations, and modifications thereof, and may be practiced or carried out in various ways. Accordingly, aspects of the pressure release assembly and fluid injector features disclosed herein are illustrative in nature and are not meant to limit the scope or application thereof. Furthermore, unless otherwise indicated, the terms and expressions employed herein have been chosen for the purpose of describing the various aspects of the pressure release assembly and fluid injector features for the convenience of the reader and are not to limit the scope thereof. In addition, it should be understood that any one or more of the components of the pressure release assembly and fluid injector features, expressions thereof, and/or examples thereof, can be combined with any one or more of the other components, expressions thereof, and/or examples thereof, without limitation.

Also, in the following description, it is to be understood that terms such as front, back, inside, outside, top, bottom, and the like are words of convenience and are not to be construed as limiting terms. Terminology used herein is not meant to be limiting insofar as devices described herein, or portions thereof, may be attached or utilized in other orientations. The various aspects of the pressure release assembly and fluid injector features will be described in more detail with reference to the drawings. As used herein, the term “proximal” means the end of a component that is positioned closer to the fluid injector and the term “distal” means the end of a component that is positioned away from the fluid injector facing end of the tubing set.

The present disclosure is drawn to a design of a pressure release assembly for use with a sealing element, such as a swabable valve, associated with a distal end of a tubing set and other fluid path elements associated with a powered fluid injector, for example a fluid injector used in medical imaging procedures for injecting one or more medical fluids. According to various embodiments, certain medical imaging procedures may include injection of a contrast media or agent that highlights certain features in the medical image. Known as contrast enhanced medical imaging, the process generally involves injection of a contrast media with a suitable flushing agent, such as saline, prior to the imaging process. Powered fluid injectors have been used to control injection of the fluids and are typically designed with one or more, commonly two, syringes for holding and dispensing the contrast media, the flushing fluid, and other medical fluids administered prior to or during the imaging procedure. For example, U.S. Application Publication Nos. 2023/0149263; 2023/0310750; and 2023/0285733; U.S. Pat. Nos. 5,383,858; 7,549,977; 9,173,995; 10,507,319, 10,583,256; and 11,712,552 and PCT Publication Nos. WO 2022/119837; and WO 2022/182235 describe front-loading syringes, tubing sets, and powered injectors in pressure jacket and jacketless examples, which disclosure is incorporated by this reference. Common contrast enhanced medical imaging procedures include computed tomography (CT), magnetic resonance imaging (MR), positron emission tomography (PET, SPECT), and angiography (CV). Due to the viscosity and need to deliver volumes of contrast during a short period of time, to provide a “tight bolus,” certain injection procedures may be performed at high injection pressures, such as pressures up to 300 psi for CT and MR, and pressures up to 1200 psi for CV procedures. The injector may be configured to inject or dispense the fluid medium contained in the first, second, and/or further syringes in a controlled manner, such as may be employed in medical procedures such as angiography, CT, PET, and NMR/MRI.

Fluid injection systems may include configurations for multi-patient injections where a portion of the disposable elements of the fluid path set, such as the pumping mechanism (syringe, components of a peristaltic pump system, etc.) and upstream components of the fluid path set are used over a series of injection procedures before disposal (multi-patient elements); and another portion of disposable elements of the fluid path set (single patient elements) which are used once with a single patient and then disposed. Thus, upon completion of a fluid injection procedure, the single patient elements are disconnected from the multi-patient elements of the fluid path and discarded; and a new, sterile single patient element is attached to the multi-patient elements for the next injection procedure. Such an arrangement may reduce expenditures per fluid injection procedure and reduce the amount of medical waste produced by an injection site. In between the injection procedures, care must be taken to ensure that the distal connection(s) of the multi-patient elements are not contaminated with microbial contaminants and are sterile when attaching the new single-patient element. Sterility of the single patient elements are ensured by storage and removal from the associated sterile packaging immediately prior to connection with the multi-patient element. Alternatively, a fluid spike may be used to access a bulk fluid container, such as a contrast container or saline bag, when filling an associated syringe with the medical fluid. In such an embodiment, the spike may need to be engaged with the distal end of the tubing set prior to filling or changed, for example when a new bulk fluid container is used. During such a process, the tubing set for filling the syringe may need to be purged of any air bubbles to prevent drawing the air bubbles into the syringe. While the air may conceivably be purged from the tubing set directly into the bulk fluid container, after attachment of the spike and spiking of the bulk fluid container, the volume of air in the tubing set may be too large for the bulk fluid container. Purging of the air through the spike into a nonsterile container risks microbial contamination of the spike and tubing set and/or fouling of surfaces with medical fluid, such as viscous and sticky contrast fluid. Thus, the present pressure release assembly may be used to purge all or substantially all of the air from the tubing set prior to spiking the bulk fluid container. If substantially all of the volume of air is purged using the pressure release assembly, as described herein, any remaining small volume of air may be purged into the bulk fluid container. The present disclosure provides components that can be utilized with the multi-patient element to ensure that the connection features of the multi-patient elements are sterile when connected with the single patient element in preparation for a subsequent injection procedure while also allowing for any air bubbles that may become entrapped in the tubing set of the multi-patient element, during the disconnection of a used single patient tubing element or fluid spike and attachment of a new single patient tubing element or fluid spike, may be substantially purged from the multi-patient tubing set so that it is not accidentally injected into a patient in the next fluid injection procedure.

According to various embodiments, the distal end of the multi-patient fluid path set having the sealing element, such as a swabable valve, attached thereto may come preassembled with the pressure release assembly according to the various embodiments of the present disclosure, with the cap in the first, non-insertion position. According to these embodiments, the pressure release assembly may act as a dust cap to prevent microbial contamination, inadvertent contact with, or soiling of the swabable valve during unpackaging and installation of the fluid path set onto the fluid injector elements. Alternatively, the pressure release assembly in the first, non-insertion position may come as a separate sterile component, which may be attached to the distal end of the multi-patient tubing set after disengagement of the previously used single patient tubing component or spike and any air entrapped in the tubing set may be purged as described herein.

The present disclosure provides embodiments of fluid path elements, fluid injector systems, and methods of using a sealing element, such as a swabable valve assembly including a pressure release assembly for fluid path elements of fluid injector tubing sets that allow for release of pressure and any volume of air during a priming operation through a swabable valve at the distal end of the tubing set. The pressure release assembly allows release of air through the outlet of the swabable valve attached to a tubing set during a priming or purging operation without release of substantial volumes of the fluid as the fluid is pressurized to prime/purge the tubing set. The pressure release assembly may include a cap removably attachable to a swabable valve assembly connected to a distal end of a tubing set.

Swabable valves typically include a proximal attachment portion for attaching the swabable valve to a tubing set, such as a distal end of a tube. The distal end of the swabable valve may include an elastomeric septum, such as a silicone end having a slit therein that fluidly seals the septum. The silicone end and slit are configured to engage a male luer fitting that may pierce the slit and provide fluid communication between the inside of the tubing and the lumen of the male luer fitting. The slit is configured to seal around the outer circumference of the male luer fitting. Alternatively, the elastomeric septum may be compressible, for example by insertion of the male luer, such that the seal between the side wall of the septum and the side wall of the valve is broken and fluid can flow around the septum into the lumen of the male luer. The swabable valve may include a female luer configuration compatible with the male luer fitting. For example, the female luer lock fitting of the swabable valve may include outer threading configured to threadably interact with inner threading on a skirt surrounding the male luer lock fitting to releasably attach the male fitting to the female fitting, for example, while or once the male luer member is piercing or has pierced the slit of the swabable valve. In certain embodiments, the pressure release assembly may be attached to the swabable valve by a friction fit between the engagement sleeve of the pressure release assembly and the outer circumference of the swabable valve or other attachment mechanism. Swabable valves are useful for replacing needle-based connector systems. In a connection procedure, the swabable valve, including the silicone septum, may be wiped or “swabbed” with an alcohol cloth or other disinfecting fluid-soaked cloth or swab to remove any microbial contaminants and provide a sterile connection surface to be pierced by a sterile male luer connector fitting. Thus, the swabable valve provides a method to ensure sterility when attaching a sterile single patient tubing component, such as a spike, having a male luer with a multi-patient tubing component having the swabable valve that has been disinfected just prior to connection. Suitable swabable valves are commercially available from Halkey-Roberts® Corp., St. Petersburg, FL, USA.

For the disposable systems in many fluid injection procedures, such as CT, CV, MR, or other imaging procedure where contrast and saline flush may be injected into a patient, there is a need to provide a sterile presentation of the swabable valve and to break the silicone seal just before priming the system. However, if the seal is broken by a male luer fitting and the luer remains in the seal for too long, for example, more than 8 hours, the silicone will take a permanent set and will not function as intended, such as will not effectively seal and prevent fluid from leaking from the tubing set. Conventional swabable valves may use a standard luer to break the seal but in certain applications it may be desirable to open up the silicone slit to allow air to purge out of the system in a purging or priming operation without attachment of a male luer connector. If the air pressure is not purged, as described herein, then the pressure in the system may build up to over 100 psi before the seal inverts and relieves itself. In such a case, not only air but possibly a volume of medical fluid may be released through the swabable valve, potentially creating a spill of medical fluid that will need to be cleaned and/or fouling various injector components with sticky and/or hazardous medical fluid.

According to various embodiments of the present disclosure, a male luer component with an open lumen and/or throughbore in the center or an irregular shape for example luer shaped pin having at least one groove on an outer surface of the pin or a pin having a cross shaped cross-section may be utilized to interrupt the seal and allow air to escape the tubing during a purging or priming operation. In certain embodiments, the fluid path may be associated with a sensor, such as a fluid or air sensor, which may detect when substantially all of the air has been purged from the fluid path and stop a purging/priming operation prior to fluid being ejected through the swabable valve or from the pressure release assembly. For certain single patient systems, the syringes will be typically filled before the disposables are attached. Once attached, all the fluid lines must be purged of air, including the tubing configured for attachment to a bulk fluid container, which include a swabable valve connecter on the end of the tubing and may be configured with a spike component having a male luer fitting. However, in certain situations the syringes may be initially filled with the medical fluid and the bulk fluid fill lines may not be used until after the initial fill, for example if there is no need for additional fluid from the bulk fluid containers until one or several additional patients. In this case, the bulk fluid fill lines cannot prime properly unless the air is allowed to escape thought the swabable valve. Alternatively, if the operator desires to refill a syringe after all lines are primed, they need to remove the dust cap (and the septum interrupter), wipe the swabable valve with a disinfecting wipe, and attach the required fluid path component, i.e., a spike, to the swabable valve and fluidly connect the bulk fluid source. Any air that may become entrapped in the tubing during this process will need to be purged prior to attachment of the new spike.

According to various embodiments herein, a dust cap that will keep the system sterile that includes a pressure release assembly may be provided in the “as shipped” state where the swabable valve is in the initial sealed configuration or “first position”. Before usage as the fluid path is being purged of any air, the operator will “crack” the swabable valve by advancing a pressure release assembly including a pressure release assembly to open the septum of the swabable valve without compromising the sterility of the system. The “cracking” of the swabable valve may be accomplished in a variety of different ways according to the various embodiments described herein. According to certain embodiments, the cracking and priming could be at least partially initiated by the operator. According to other embodiments, the cracking and purging/priming could be initiated by a component that interfaces with the fluid injector (fluid control module or other controller) and the fluid injector could activate the purging/priming sequence as part of an injector setup sequence-either an initial setup sequence or new patient setup sequence after a prior patient injection procedure. In certain embodiments, the pressure release assembly and swabable valve assembly may be configured so that it only fits into a fluid control module after it is activated. In other embodiments, the pressure release assembly and swabable valve assembly may be manually activated by the user. In still other embodiments, the pressure release assembly and swabable valve assembly may be inserted into a cassette associated with the fluid injector, wherein insertion into the cassette either activates the a assembly or allows activation of the assembly by a controller associated with the fluid injector, for example using one or more sensors to determine a status of the pressure release assembly and swabable valve assembly and moving the pressure release assembly from the first, non-insertion position to the second, insertion position.

According to various embodiments, the release assembly may thread onto the standard female luer thread of the swabable valve, and the pressure release assembly may be advanced by pushing a center pin from a first, non-insertion position to a second insertion position where the center pin has pierced or opened the seal of the swabable valve. In other embodiments, the pressure release assembly may be a threaded pin that interfaces with the standard female luer thread (or have a unique thread) and the pressure release assembly may be advanced by turning the threaded pin and advancing the pin through the seal of the swabable valve. In certain embodiments, there may be a line or mark to indicate the pressure release assembly is advanced to the correct position.

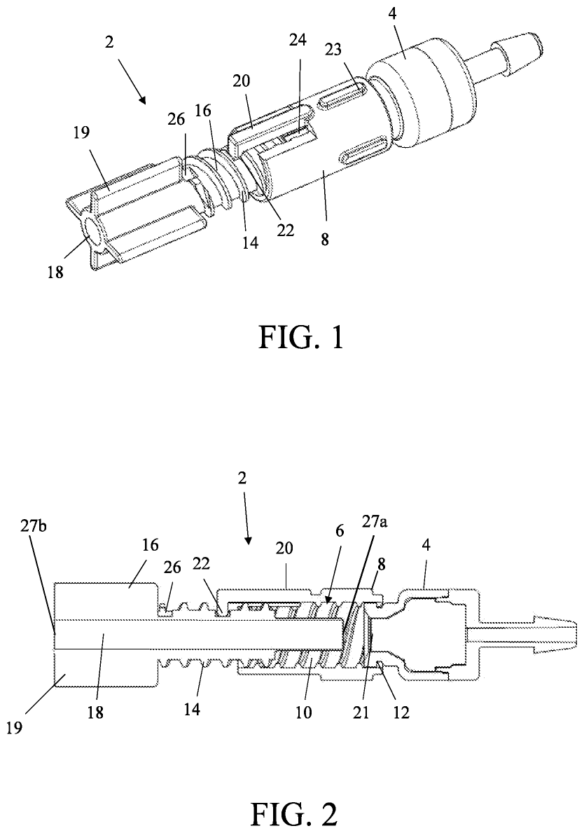

According to one non-limiting embodiment or aspect of the present disclosure, as illustrated in FIGS. 1-4, a pressure release assembly 2 is shown and described in detail. The pressure release assembly 2 may be reversibly threadably attachable to a swabable valve 4. In one example of the present disclosure, the pressure release assembly 2 may include a connection arrangement 6, provided on an inner surface of an engagement sleeve 8 of the pressure release assembly 2. In one example, the engagement sleeve 8 may have a generally cylindrical shape. A cavity 10 may be defined by the engagement sleeve 8. In one non-limiting embodiment or aspect of the present disclosure, the connection arrangement 6 may include threading on the inner surface of the engagement sleeve 8 configured to engage corresponding threading 12 on the swabable valve 4 to secure the pressure release assembly 2 to the swabable valve 4. It is to be understood the threading of the engagement sleeve 8 and the threading 12 of the swabable valve 4 may be corresponding male and female threadings. In one example, a distal portion of the swabable valve 4 may be received in a proximal end of the engagement sleeve 8. In this example, the threading 12 of the swabable valve 4 is a male threading that is received in female threading of the connection arrangement 6 of the engagement sleeve 8.

In one non-limiting embodiment or aspect of the present disclosure, the connection arrangement 6 of the engagement sleeve 8 may further engage a threading 14 provided on a pin member 16 held within the cavity 10 of the engagement sleeve 8. It is to be understood that an alternative connection arrangement or method other than threading may also be used to operatively connect the pin member 16 to the engagement sleeve 8. In one example, the pin member 16 may be rotated in a first direction to advance the pin member 16 relative to the engagement sleeve 8 in a proximal direction (towards the swabable valve 4) and may be rotated in a second direction (opposite to the first direction) to advance the pin member 16 relative to the engagement sleeve 8 in a distal direction (away from the swabable valve 4). In another embodiment, once the pin member 16 is moved to the second, insertion position, the pin member 16 cannot be moved back to the first, non-insertion position. In this embodiment, the engagement sleeve 8 must be removed from the swabable valve 4 by rotating the engagement sleeve 8 relative to the swabable valve 4 to separate the pressure release assembly 2 from the swabable valve 4.

In one example, the pin member 16 may define an at least partial throughbore 18. According to a first embodiment, the at least partial throughbore 18 may be a complete throughbore extending from a proximal end 27a to a distal end 27b of the pin member 16 along the longitudinal axis of the pin member 16. In a second embodiment, the at least partial throughbore 18 may be a partial throughbore extending from the proximal end 27a of the pin member 16. In one non-limiting embodiment or aspect of the present disclosure, a plurality of gripping members 19 may be provided on an outer surface of the distal end 27b of the pin member 16. A user may grasp the plurality of gripping members 19 to assist in rotating or advancing the pin member 16 relative to the engagement sleeve 8. In one example, the plurality of gripping members 19 may be fins or protrusions distributed parallel to the longitudinal axis about the circumference of the distal end 27b of the pin member 16. In one non-limiting embodiment or aspect of the present disclosure, a plurality of gripping members 23 may be provided on an outer surface of the engagement sleeve 8. A user may grasp the plurality of gripping members 23 to assist in rotating or advancing the engagement sleeve 8 relative to the swabable valve 4.

With continued reference to FIGS. 1-4 according to a non-limiting embodiment of the present disclosure, the engagement sleeve 8 may include a flexible retaining arm 20 protruding distally (shown) or proximally (not shown) therefrom and configured to engage the outer surface of the pin member 16 when the pin member 16 is in one of a first, non-insertion position in which the swabable valve 4 may be shipped having the seal 21 in a sealed state and a second, insertion position where the pin member 16 opens a seal 21 of the swabable valve 4. When the flexible retaining arm 20 engages the outer surface of the pin member 16 in the second position, according to certain embodiments, the pin member 16 may be locked in the second, insertion position—i.e., the seal 21 of swabable valve 4 may be locked in an open position with the pin member 16 inserted therethrough. To fluidly seal the seal 21 of swabable valve 4, the pressure release assembly 2 must be disengaged from the swabable valve 4.

In use, the pressure release assembly 2 may be shipped in sterile packaging in the first, non-insertion position, in which the pin member 16 is in the distal position. In this example of the pressure release assembly 2, the retaining arm 20 may be snapped into a first detent 22 on the threading 14 of the pin member 16 at the first, non-insertion position. In certain embodiments, the pressure release assembly 2 may be shipped with the associated swabable valve 4 and the entire pressure release assembly 2 and swabable valve 4 may be attached to the tubing and attached to the fluid injector fluid path. In other embodiments, the swabable valve 4 may be previously attached to the fluid injector fluid path, the swabable valve 4 may be disinfected, for example by swabbing with a disinfecting cloth, as described herein, and the pressure release assembly 2 may be removed from sterile packaging and threadably installed onto the swabable valve 4.

In one example of the present disclosure, the throughbore 18 of the pin member 16 may allow air from the inner lumen of the tubing connected to the swabable valve 4 to be released through the swabable valve 4 and the throughbore 18 of the pin member 16 when the pin member 16 is moved to the second, insertion position and the fluid injector is instructed to perform a purging or priming operation where the fluid and/or air inside the tubing is pressurized, for example by moving a piston and plunger of a syringe in the distal direction. In certain embodiments, the throughbore 18 of the pin member 16 may further include an adsorbent material or filter material configured to absorb or prevent passage of any medical fluid that may be expelled during a purging/priming sequence while allowing air to pass therethrough. Non-limiting examples of such filter material include, Porex (porous sintered polytetrafluoroethylene (PTFE)) based filter materials, membranes, filters, and fibers; and other materials that are pervious to air or other gas but substantially impervious to the passage of a liquid such as a contrast agent or saline.

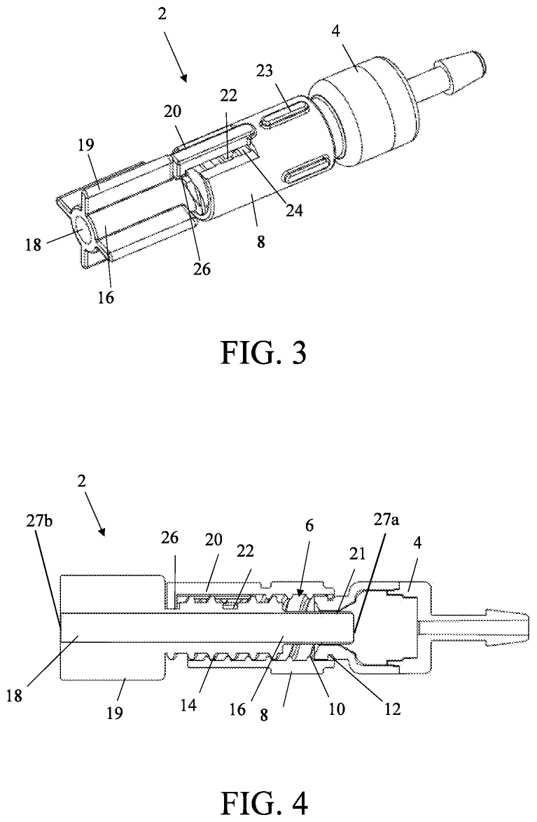

In certain embodiments, as shown in FIGS. 1 and 3, the engagement sleeve 8 of the pressure release assembly 2 may also define one or more vents 24 on a sidewall of the engagement sleeve 8. The one or more exit vents 24 may allow any air from the tubing that exits through the seal 21 of the swabable valve 4 outside of the pin member 16 to escape from the engagement sleeve 8 and may further assist by permitting the retaining arm 20 to flex circumferentially about the outer surface of the pin member 16. It is to be understood that the flexing motion of the retaining arm 20 may be minimal but may occur as the pin member 16 is inserted into the engagement sleeve 8.

During a purging or priming sequence of the tubing connected to the swabable valve 4, the injector syringe and tubing may be pressurized. Prior to or during the purging or priming sequence, the pressure release assembly 2 may be moved from the first non-insertion position to the second, insertion position. Moving from the first position to the second position may be manually accomplished by the user gripping the one or more of the plurality of gripping members 19 at the distal end of the pin member 16 and rotating the pin member 16 in the clockwise or counterclockwise direction. Depending on the directionality of the threading, in certain embodiments, moving the pin member 16 from the first position to the second position may be accomplished by rotating the pin member 16 in the counterclockwise direction. As the pin member 16 is rotated, the threading 14 moves the pin member 16 in the proximal direction until the proximal end 27a of the pin member 16 engages and breaks the seal 21 associated with the swabable valve 4 (e.g., by piercing through the slit in the elastomeric sealing material) and is in the second, insertion position. Once the seal 21 of the swabable valve 4 is broken, the throughbore 18 of the pin member 16 is in fluid communication with the inner volume of the tubing. As the purging and priming sequence continues, air from the inner volume of the tubing is purged out of the tubing and through the throughbore 18 of the pin member 16. At the end of the purging and priming sequence, the pin member 16 may be moved from the second, insertion position back to the first non-insertion position and the swabable valve 4 returns to the sealed configuration. The pin member 16 may be moved from the second position to the first position by rotating the plurality of gripping elements 19 and the pin member 16 in the opposite direction from the direction it was rotated to move from the first position to the second position, i.e., counter-clockwise if the pin member 16 was originally rotated clockwise to move from the first position to the second position; or clockwise if the pin member 16 was originally rotated counter-clockwise to move from the first position to the second position. As the pressure release assembly 2 is moved from the first position to the second position, the retaining arm 20 may move from the first detent 22 holding the pin member 16 in the first position to a second detent 26, holding the pin member 16 in the second, insertion position. As the retaining arm 20 moves into the second detent 26, an audible click may be heard by the user or a tactile click may be felt by the user to indicate that the pressure release assembly 2 is now in the second, insertion position and the purging/priming operation may proceed to remove air from the tubing. According to another embodiment, when the pin member 16 is moved into the second, insertion position, the retaining arm 20 may engaged the second detent 26 in a non-removable engagement. In this embodiment, once the tubing has been substantially purged of air and the single patient component or spike is ready to be installed, the user may simply disengage the entire pressure release assembly 2 from swabable valve 4, such that the seal of the swabable valve 4 is sealably reformed and ready to be swabbed and the single patient component or spike to be installed.

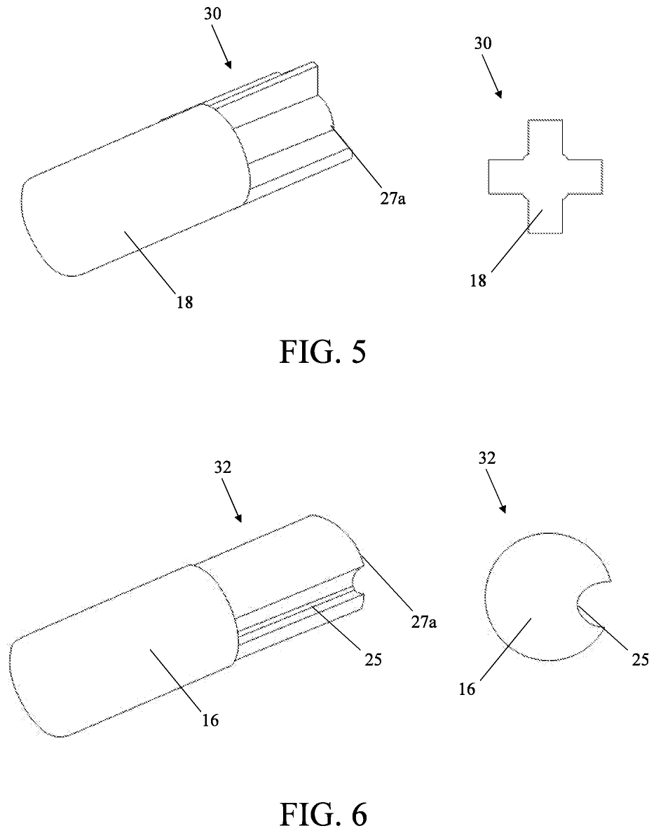

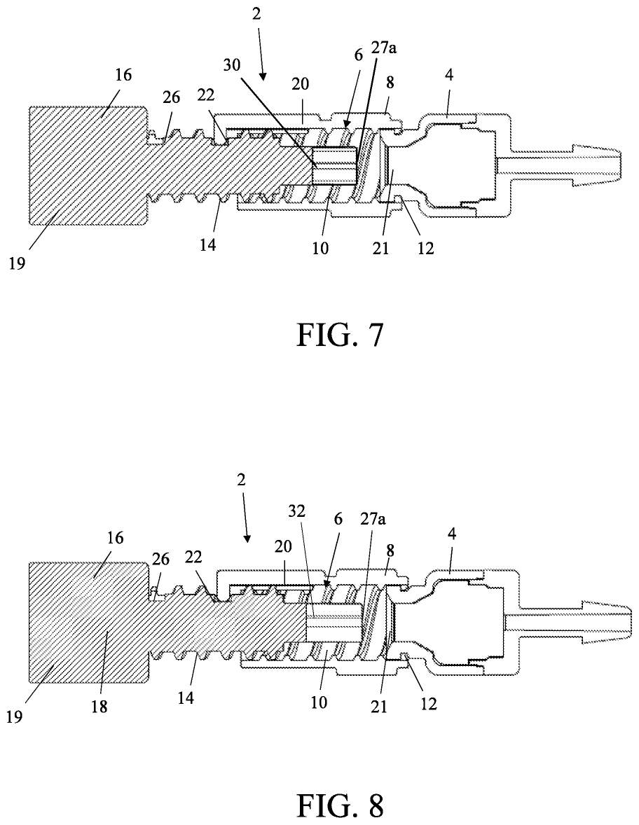

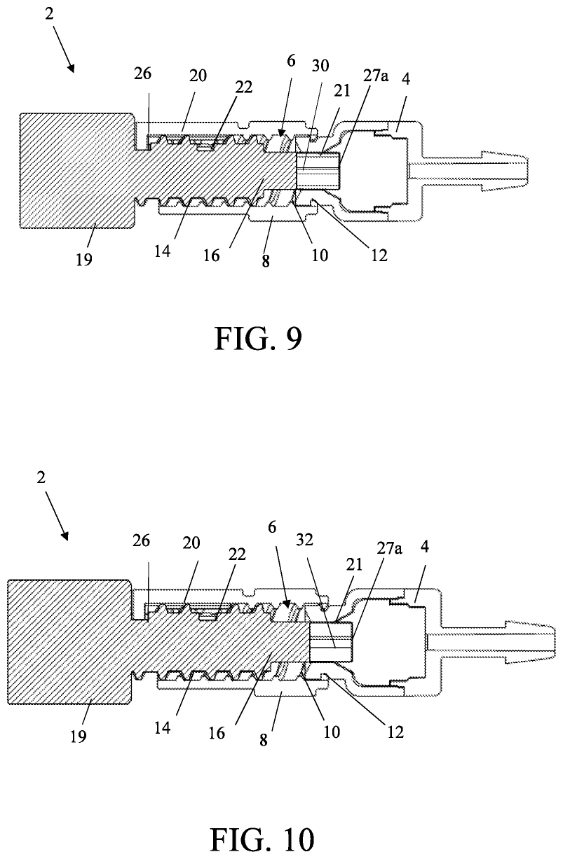

As shown in FIGS. 5-10, according to non-limiting embodiments of the present disclosure, at least a portion of the pin member 16 may have a cross-sectional shape other than the cylindrical shape illustrated in FIGS. 1-4. For example, as illustrated in FIG. 5, the proximal end 27a of the pin member 16 may include a cross-shaped cross-section 30. As shown in FIGS. 6-10, the proximal end 27a of the pin member 16 may include a moon-shaped cross-section 32 defined by at least one longitudinal groove 25 defined in a side of the pin member 16. These different shapes for the pin member 16 may be provided to assist in releasing air from the swabable valve 4 and through the release assembly 2. In these examples, the pin member 16 may not define a throughbore for releasing the air from the swabable valve 4. Instead, as shown in FIGS. 9 and 10, after the pin member 16 has been inserted into the swabable valve 4 and broken the seal 21 of the swabable valve 4, the proximal end 27a of the pin member 16 may hold open the seal 21 of the swabable valve 4. Air from the swabable valve 4 may be released through the grooves defined on the sides of the pin member 16 by the cross-sectional shape of the proximal end 27a of the pin member 16. The air can then be released from the release assembly 2, for example, through the threading of the engagement sleeve 8 and/or the vents 24 defined in the engagement sleeve 8. In one example, it is also contemplated that a partial throughbore may be defined in the distal end 27b of pin member 16, which will assist in reducing material costs (i.e., less plastic) associated with forming pin member 16.

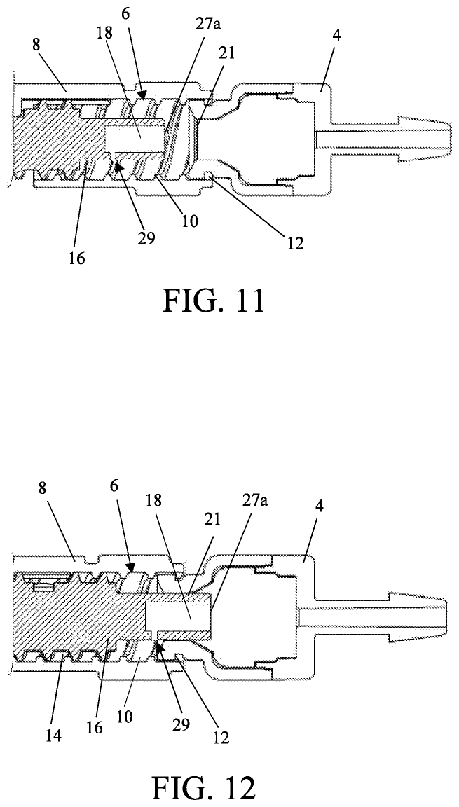

According to one non-limiting embodiment or aspect of the present disclosure, as illustrated in FIGS. 11 and 12, the pin member 16 may define a partial throughbore 18 in the proximal end 27a of the pin member 16. Furthermore, at least one vent 29 may be defined in a side surface of the proximal end 27a of the pin member 16 connecting the partial throughbore 18 to the cavity 10 of the engagement sleeve 8. It is also to be understood that additional vents may be defined in the proximal end 27a of the pin member 16. The vent 29 may include an inlet defined in an inner surface of the pin member 16 and an outlet defined in an outer surface of the pin member 16. As shown in FIG. 11, when the pin member 16 is positioned in the first, non-insertion position, the vent 29 are positioned distally of the seal 21 of the swabable valve 4. In this configuration, the vent 29 is protected from particulate or microbial contamination as it is positioned within the cavity 10 of the engagement sleeve. As shown in FIG. 12, when the pin member 16 is positioned in the second, insertion position, the inlet at the proximal end 27a of the partial throughbore 18 is positioned proximal to the seal 21 of the swabable valve 4, and the vent 29 remains positioned distally from the seal 21 of the swabable valve 4. Therefore, when the pin member 16 is positioned in the second, insertion position, air can be released from the swabable valve 4 through the inlet at the proximal end 27a of the partial throughbore 18 and out of the vent 29 to be released from the engagement sleeve 8 to atmosphere.

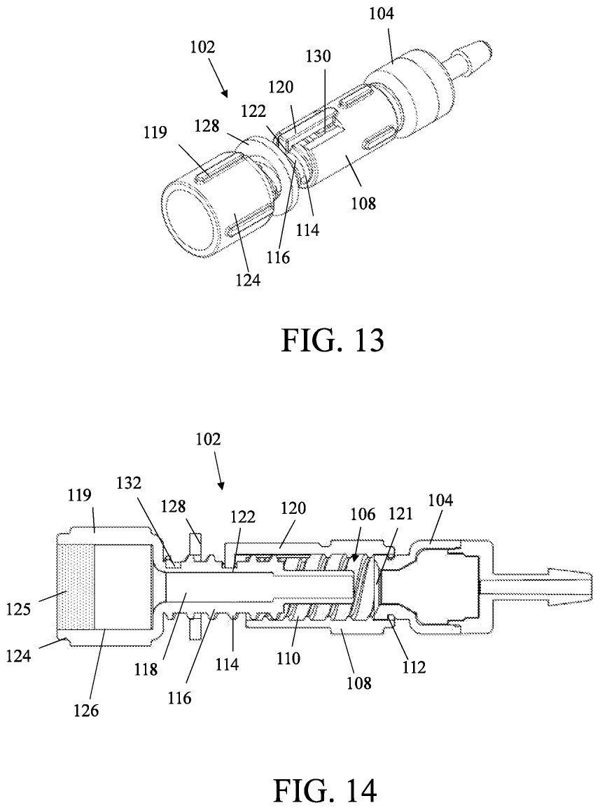

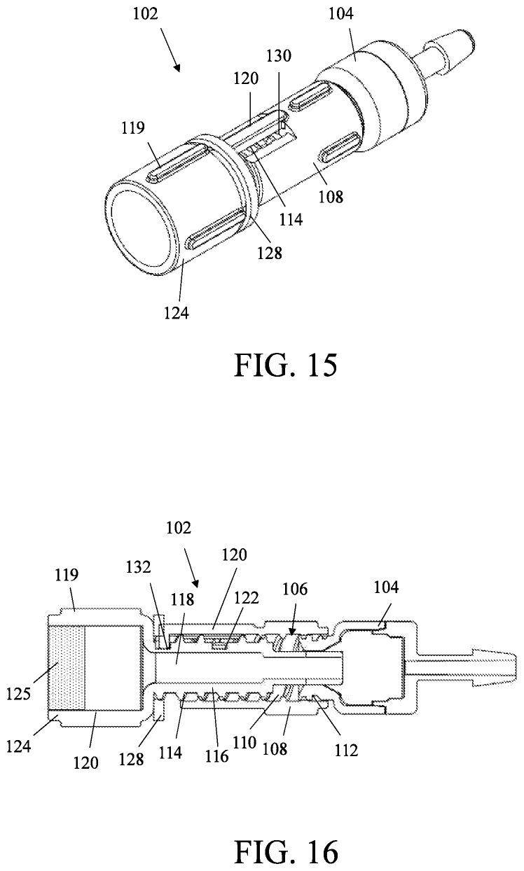

According to one non-limiting embodiment or aspect of the present disclosure, as illustrated in FIGS. 13-16, a pressure release assembly 102 is shown and described in detail. The pressure release assembly 102 may be reversibly threadably attachable to a swabable valve 104 as previously described with reference to FIGS. 1-4. It is to be understood that any features corresponding to those in FIGS. 1-4 will not be discussed again in detail herein.

In one example, the pin member 116 may define a throughbore 118. The throughbore 118 may extend from the proximal end of the pin member 116 to the distal end of the pin member 116 along the longitudinal axis of pin member 116.

In one example of the present disclosure, the throughbore 118 of the pin member 116 may allow air from the inner lumen of the tubing connected to the swabable valve 104 to be released through the swabable valve 104 and the throughbore 118 of the pin member 116 when the pin member 116 is moved from the first, non-insertion position to the second, insertion position and the fluid injector is instructed to perform a purging or priming operation where the fluid inside the tubing is pressurized, for example by moving a piston and plunger of a syringe in the distal direction. In the present embodiment or aspect, the pin member 116 may include a bell element 124 at the distal end of the pin member 116, wherein the bell element 124 defines an internal volume 126. According to these embodiments, the bell element 124 may be configured to capture or secure small volumes of fluid that may be expelled through the pressure release assembly 102 during a purging or priming sequence. This may prevent fluid contamination or fouling by retaining any medical fluid with the pressure release assembly 102. In certain embodiments, the bell element 124 may include an adsorbent material or filter material 125 to absorb or prevent escape of the medical fluid while allowing any air to pass therethrough. For example, the adsorbent material may be a material that is pervious to air but substantially impervious to liquid, such as a Porex based material as described herein, such that air may pass through the filter material 125 whereas contrast or saline liquid may not pass through the filter material 125. In other embodiments, the bell element 124 may include one or more gripping elements 119 protruding from an outer surface of the bell element 124 to allow a user to rotate the pin member 116 to move the pin member 116 from the first position to the second position, and in certain embodiments, from the second position back to the first position.

Certain embodiments of the various pressure release assembly 102 described herein may further include one or more indicators, such as a visual band 128, for example on one or both of the pin member 116 and the engagement sleeve 108, that may indicate to the user that the pin member 116 has not yet been completely moved to the second, insertion position. As the pin member 116 is rotated, the retaining arm 120 of the engagement sleeve 108 may engage the visual band 128 and distally move the visual band 128 and the pin member 116 is rotated. In the second, insertion position, the visual band 128 may be moved completely about the proximal wall of the bell element 124, indicating to the user that the pressure release assembly 102 is ready for initiation of the purging/priming sequence. Alternatively, the visual band 128 may be at least one colored ring or at least a portion of a barcode around the circumference of the pin member 116, wherein the visual band 128 may be positioned such that when the pin member 116 is in the second, inserted position, the colored ring is in a particular location, such as fully within the engagement sleeve 108 and not visible to a user or at a position where a sensor on the fluid injector can sense the location of the at least one colored ring or the at least a portion of the barcode and send a signal to the controller of the fluid injector that the pin member is in the second position.

During a purging or priming sequence of the tubing connected to the swabable valve 104, the injector syringe, and other tubing portions may be pressurized. Prior to or during the purging or priming sequence, the pressure release assembly 102 may be moved from the first non-insertion position to the second, insertion position. Moving from the first position to the second position may be manually accomplished by the user gripping the one or more gripping members 119 at the bell feature 124 of the pin member 116 and rotating the one or more gripping member 119 and pin member 116 in the clockwise direction. Depending on the directionality of the threading, in certain embodiments, moving the pin member 116 from the first position to the second position may be accomplished by rotating the one or more gripping members 119 and the pin member 116 in one of the clockwise or counterclockwise direction, depending on the direction of the threading. As the pin member 116 is rotated, the threading 114 moves the pin member 116 in the proximal direction until the proximal end of the pin member 116 engages and breaks the seal 121 associated with the swabable valve 104 and is in the second, insertion position. Once the seal 121 of the swabable valve 104 is broken, the lumen or throughbore 118 of the pin member 116 is in fluid communication with the inner volume of the tubing. As the purging and priming sequence continues, air from the inner volume of the tubing is purged out of the tubing and through the throughbore 118 of the pin member 116 and into the bell element 124; and if the filter material 125 is present, the air exits through the filter material 125. Any liquid that may also be purged or primed through throughbore 118 and collect in the bell element 124. In embodiments, where the filter material 125 is present, the filter material 125 may prevent any liquid from escaping the bell element 124. At the end of the purging and priming sequence, the pin member 116 may be moved from the second, insertion position back to the first non-insertion position and the swabable valve 104 returns to the sealed configuration. The pin member 116 may be moved from the second position to the first position by rotating the one or more gripping elements 119 and the pin member 116 in the opposite direction from the direction it was rotated to move from the first position to the second position, i.e., counter-clockwise if the pin member 116 was originally rotated clockwise to move from the first position to the second position; or clockwise if the pin member 116 was originally rotated counter-clockwise to move from the first position to the second position. As the pressure release assembly 102 is moved from the first position to the second position, the retaining arm 120 may move from the first detent 122 holding the pin member 116 in the first position to a second detent 132, holding the pin member 116 in the second, insertion position. As the retaining arm 120 moves into the second detent 132, an audible click may be heard by the user or a tactile click may be felt by the user to indicate that the pressure release assembly 102 is now in the second, insertion position and the purging/priming operation may proceed to remove air from the tubing. Alternatively, in embodiments where the pin member 116 is locked in the second position, the pin member 116 may be removed from the broken seal 121 by removing the pressure equalizing cap 102 from the swabable valve 104.

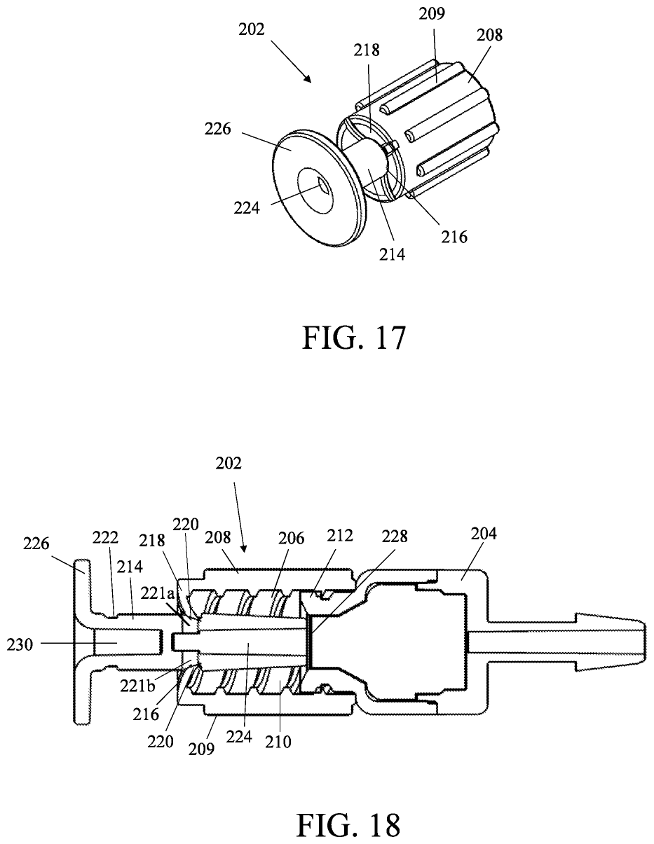

According to one non-limiting embodiment of the present disclosure, as illustrated in FIGS. 17-19, a pressure release assembly 202 is shown and described in detail. The pressure release assembly 202 may be reversibly threadably attachable to a swabable valve 204. In one example of the present disclosure, the pressure release assembly 202 may include a connection arrangement 206 provided on an inner surface of an engagement sleeve 208 of the pressure release assembly 202. In one example, the engagement sleeve 208 may have a generally cylindrical shape. A plurality of gripping elements 209 may be positioned on an outer circumferential surface of the engagement sleeve 208 to assist a user in grasping the engagement sleeve 208 for rotating the engagement sleeve 208 relative to the swabable valve 204. A cavity 210 may be defined by the engagement sleeve 208. In one non-limiting embodiment or aspect of the present disclosure, the connection arrangement 206 may include threading on the inner surface of the engagement sleeve 208 configured to engage threading 212 on the swabable valve 204 to secure the pressure release assembly 202 to the swabable valve 204. It is to be understood the threading of the engagement sleeve 208 and the threading 212 of the swabable valve 204 may be corresponding male and female threadings. In one example, a distal portion of the swabable valve 204 may be received in a proximal end of the engagement sleeve 208. In this example, the threading 212 of the swabable valve 204 is a male threading that is received in female threading of the connection arrangement 206 of the engagement sleeve 208. In certain embodiments, the pressure release assembly 202 may be attached to the swabable valve 204 by a friction fit between the inner surface of the engagement sleeve 208 and the outer circumference of the swabable valve 204 or by some other suitable attachment mechanism known in the art. These alternative attachment means may be incorporated into any of the various embodiments of the pressure release assemblies described herein.

In one non-limiting embodiment or aspect of the present disclosure, the pressure release assembly 202 may include a pin member 214 that is slidably moveable from a first non-insertion position to a second, insertion position relative to the engagement sleeve 208. The engagement sleeve 208 may define an opening 216 on a distal surface thereof. The pin member 214 may be received in the opening 216 to slidably move relative to the engagement sleeve 208. One or more flexible engagement elements 218 may extend radially inward from the opening 216 to flexibly engage one or more detent 220, 222 defined on the slidable pin member 214. The pin member 214 may include an at least partial throughbore or lumen 224 to provide fluid communication between the proximal end and the exterior of the pin member 214. In one embodiment, where the pin member 214 includes a partial throughbore 224, fluid communication is provided between the proximal end of the pin member 214 and at least one side vent 221a, 221b positioned at the distal end of the partial throughbore 224. The at least one side vent 221a, 221b may be located at a position on the pin member 214 that is enclosed by the engagement sleeve 208. According to this embodiment, the position of the at least one side vent 221a, 221b may be selected to prevent microbial contamination of the partial throughbore 224, for example by inadvertent touching of the at least one side vent 221a, 221b by a finger of contaminated surface. In other embodiments, the pin member 214 may include a full throughbore extending along the longitudinal axis from a proximal end to the distal end of pin member 214. The distal end of the pin member 214 may include a flange 226 configured for pressing engagement, for example by manually pressing the pin member 214 to move from the first, non-insertion position to the second, insertion position or by pressing by an electromechanical member of a cassette element to move the pin member 214 from the first, non-insertion position to the second, insertion position.

The pin member 214 may further include a first circumferential detent 220 and, in certain embodiments, a second circumferential detent 222 configured for interacting with the one or more flexible engagement elements 218 to retain the pin member 214 in the first position and the second position, respectively. The first circumferential detent 220 may be located around an approximately central portion of the pin member 214 to releasably retain the pin member 214 in the first position, and the second circumferential detent 222 may be located adjacent the proximal surface of the flange 226 to releasably or non-releasably retain the pin member 214 in the second position. In one example, the pin member 214 may be pushed in a first direction to advance the pin member 214 relative to the engagement sleeve 208 in a proximal direction (towards the swabable valve 204) and, in embodiments where the pin member 214 is releasably retained in the second position, may be pulled in a second direction (opposite to the first direction) to advance the pin member 214 relative to the engagement sleeve 208 in a distal direction (away from the swabable valve 204) to disengage the pin member 214 from the seal 228 of the swabable valve 204. The first, non-insertion position may be understood to be a position in which the pin member 214 does not crack or open a seal 228 of the swabable valve 204, and the second, insertion position may be understood to be a position in which the pin member 214 has cracked and is inserted into the swabable valve 204 through the seal 228. In another embodiment, once the pin member 214 is pushed to the second, insertion position, the pin member 214 is non-releasably retained in the second, insertion position and cannot be moved back to the first, non-insertion position. In this embodiment, the engagement sleeve 208 must be removed from the swabable valve 204 by rotating the engagement sleeve 208 relative to the swabable valve 204 to separate the pressure release assembly 202 and pin member 214 from the swabable valve 204.

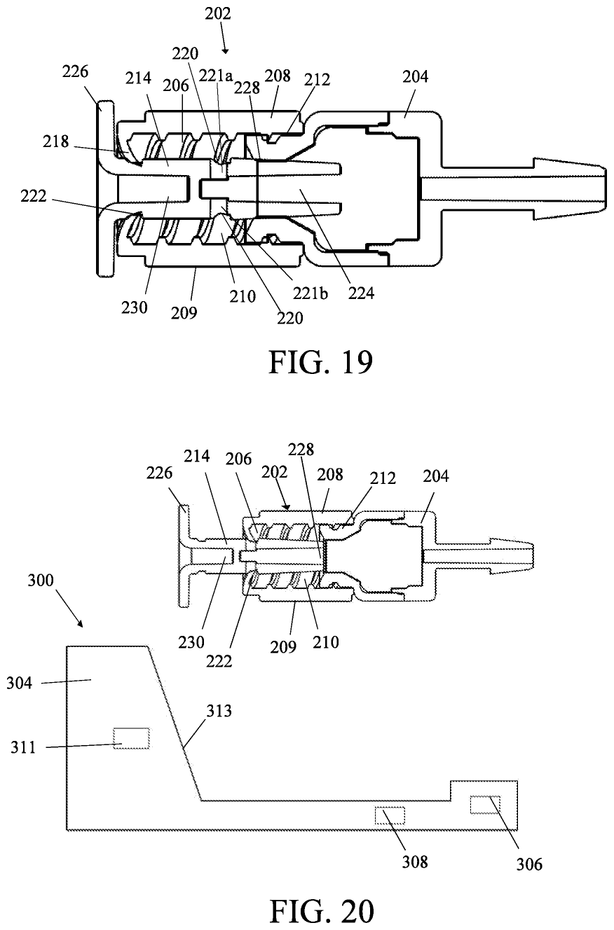

According to other embodiments, as illustrated in FIGS. 18 and 19, the pressure release assembly 202 of the present disclosure may optionally include an adsorbent material or filter material 230, as described herein, held near the distal end of the pin member 214 within the longitudinal throughbore or lumen 224 extending from the proximal end to the distal end of the pin member 214. In the second, insertion position, air may pass through the throughbore or lumen 224 during a purging/priming operation of the tubing operatively connected to the swabable valve 204. However, any small volumes of medical liquid that may inadvertently flow through the swabable valve 204 may be retained by the adsorbent material or filter material 230.

In use, the pressure release assembly 202 may be shipped in sterile packaging in the first, non-insertion position, in which the pin member 214 is in the distal position separated from and not inserted through the swabable valve 204. In this example of the pressure release assembly 202, the one or more flexible engagement elements 218 may be snapped into or positioned in the first detent 220 on the pin member 214 at the first, non-insertion position. In certain embodiments, the pressure release assembly 202 may be shipped with the associated swabable valve 204; and the entire pressure release assembly 202 and swabable valve 204 may be previously attached to the tubing. In other embodiments, the swabable valve 204 may be previously attached to the associated tubing of fluid path, the swabable valve 204 may be disinfected, for example by swabbing with a disinfecting cloth, as described herein, and the pressure release assembly 202 may be removed from sterile packaging and threadably installed onto the swabable valve assembly 204. In certain embodiments, a previously used pressure release assembly 202 may be removed from the swabable valve 204 and a new, sterile pressure release assembly 202 may be installed on the swabable valve, for example after swabbing disinfection of swabable valve 204.

In one example of the present disclosure, the throughbore 224 of the pin member 214 may allow substantially all of a volume of air in the inner lumen of the tubing connected to the swabable valve 204 to be released through the swabable valve 204 and the at least partial throughbore 224 of the pin member 214 when the pin member 214 is moved from the first, non-insertion position to the second, insertion position and the fluid injector is instructed to perform a purging or priming operation where the fluid inside the tubing is pressurized, for example by moving a piston and plunger of a syringe in the distal direction. To activate the pressure release assembly 202, the user may press the distal surface of the flange 226 to slide the pin member 214 in the proximal direction relative to the engagement sleeve 208 prior to or during the purging or priming operation by the fluid injector. Upon application of pressure, the flexible engagement elements 218 of the engagement sleeve 208 release from the first circumferential detent 220 on the pin member 214 and the pin member 214 slides proximally relative to the engagement sleeve 208, piercing the seal 228 of the swabable valve 204 and providing fluid communication between the at least partial throughbore or lumen 224 of the pin member 214 and the lumen of the fluid path (i.e., the tubing) attached to the swabable valve 204. In certain embodiments, the pin member 214 continues to slide proximally relative to the engagement sleeve 208 until the one or more flexible engagement elements 218 of the engagement sleeve 208 engage the second circumferential detent 222, holding the pressure release assembly 202 in the second, insertion position. Alternatively in other embodiments, the pin member 214 slides until a proximal surface of the flange 226 of the pin member 214 abuts a distal surface of the engagement sleeve 208. In certain embodiments, the pressure release assembly 202 may be returned to the first position by pulling the flange 226 in the distal direction relative to the engagement sleeve 208. Alternatively, in embodiments where the pin is non-releasably retained in the second, insertion position, rotational removal of the engagement sleeve 208 from the swabable valve 204 removes the pin member 214 from the seal 228 of the swabable valve 204, thereby re-sealing the swabable valve 204. After removal of the pressure release assembly 202 from the swabable valve 204, tubing elements, such as a spike, having a male luer fitting may be engaged with swabable valve 204, after suitable disinfection of swabable valve 204 (e.g., swabbing the swabable valve with a swabbing, disinfection wipe).

Other embodiments of the pressure release assembly may be substantially similar, having a sliding activation mechanism to go from the first, non-insertion position to the second, insertion position. However, according to these embodiments, the flange 226 on the pin member 214 may be replaced by a bell element (see, bell element 124 associated with FIGS. 13-16) configured to catch and retain any medical fluid, as described herein, that may inadvertently pass through the swabable valve during the purging/priming sequence. In certain embodiments, the interior volume of the bell element may include an adsorbent material to adsorb any medical liquid; or the distal end of the bell element may include an air pervious but substantially liquid impervious filter material, as described herein, configured to allow air to pass therethrough but retain any medical liquid that contacts the material.

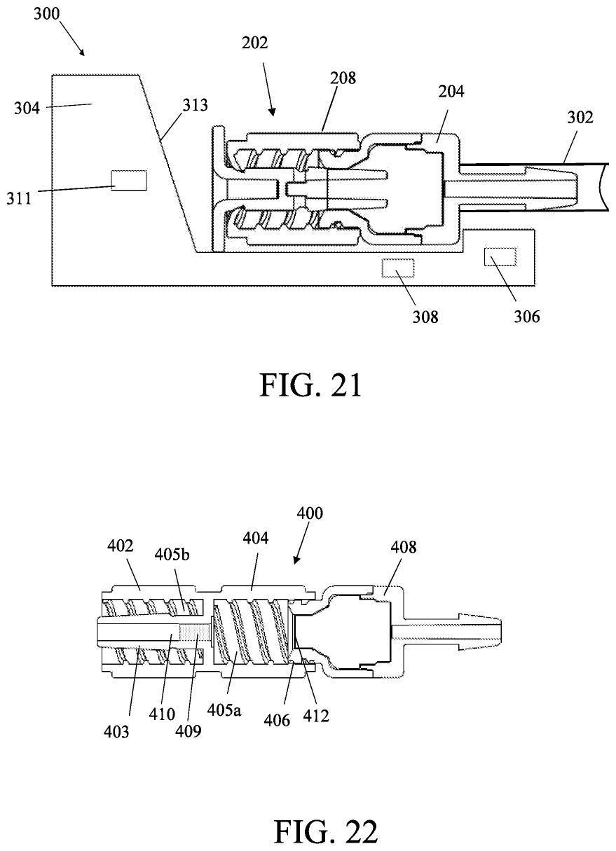

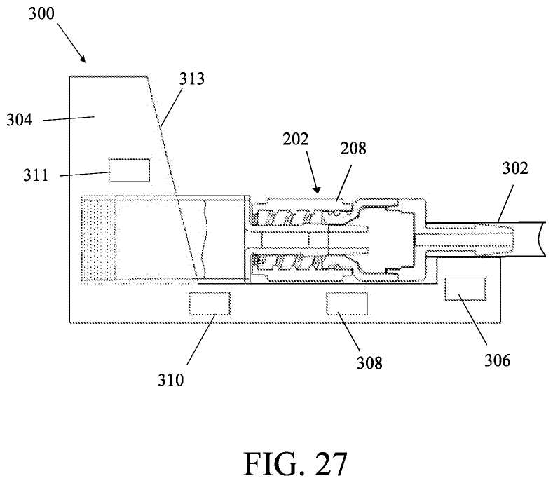

As shown in FIGS. 20, 21, and 27, according to other non-limiting embodiments or aspects of the present disclosure, the pressure release assembly according to the various embodiments described herein, for example, the pressure release assembly 202 and associated swabable valve 204 may be configured to interact with and engage one or more features of a fluid injector module or cassette 300. While described with relation to the pressure release assembly 202/swabable valve 204 embodiment, it is also to be understood that the pressure release assemblies 2, 102, 400, 502/swabable valve 4, 104, 408, 504 embodiments may also be used in conjunction with the fluid injector module or cassette 300 as described herein. For example, tubing 302, swabable valve 204, and pressure release assembly 202 may be configured to be inserted into one or more fluid injector constraints 304. According to certain embodiments, the orientation and shape of the one or more injector constraints 304 may require that the pressure release assembly 202 may be moved from the first, non-insertion position to the second, insertion position prior to installation of the assembly into the one or more injector constraints 304. According to these embodiments, the user is required to activate the pressure release assembly 202 during assembly of the fluid path assembly with the injector module 300. This ensures that the pressure release assembly 202 is in the second, insertion position upon installation and that the fluid path is ready for an air purging operation. In another embodiment, the injector module 300 may include an activation surface (for example a slanted activation surface 313), that moves the pin element 214 from the first, non-insertion position to the second, insertion position, as the pressure release assembly 202/swabable valve 204 assembly is inserted into the injector module 300. In another embodiment, an electromechanical component 311 of the injector module 300, such as a solenoid valve, in communication with a controller of the fluid injector may activate the pressure release assembly 202 once the pressure release assembly 202/swabable valve 204 assembly is inserted into the injector module 300 and the fluid injector controller activates a priming or purging operation. As described herein, the injector module 300 may further include one or more sensors 306, 308, 310. For example, a first sensor 306 may be a liquid or air sensor configured to detect when the tubing set 302 is substantially purged of air, e.g., by detecting liquid or an air/liquid interface immediately proximal to the swabable valve 204. The first sensor 306 may then signal the injector module 300 that the purging operation is complete, and the fluid path is essentially purged of any air. In other embodiments, the fluid injector module 300 may include a second sensor 308 configured to sense when the pressure release assembly 202 has been installed within the injector constraints 304. The second sensor 308 may then send a signal to a controller of the injector module 300 that the seal 228 of the swabable valve 204 has been broken and that the fluid path 302 is ready for the purging of air therefrom. In certain embodiments, the injector module 300 may include a third sensor 310, such as a liquid or air sensor configured to detect a liquid/air interface within the pin element 214 or optional bell element (see FIG. 27), to determine that substantially all of a volume of air has been purged or primed from the tubing 302. Once the sensor 306, 310 has determined that substantially all of the volume of air has been purged from the fluid path 302, the pressure release assembly 202/swabable valve 204 assembly may be removed from the injector module 300, the pressure release assembly 202 may be removed from the swabable valve 204, the swabable valve 204 may be disinfected by swabbing with a disinfecting cloth, and a fluid path element may be installed on the swabable valve 204. For example, a spike may be installed on the swabable valve 204 and inserted into a bulk fluid container; and any residual volume of air may be purged into the fluid container prior to drawing fluid into the syringe (or peristaltic pump) through the spike and fluid path 302.

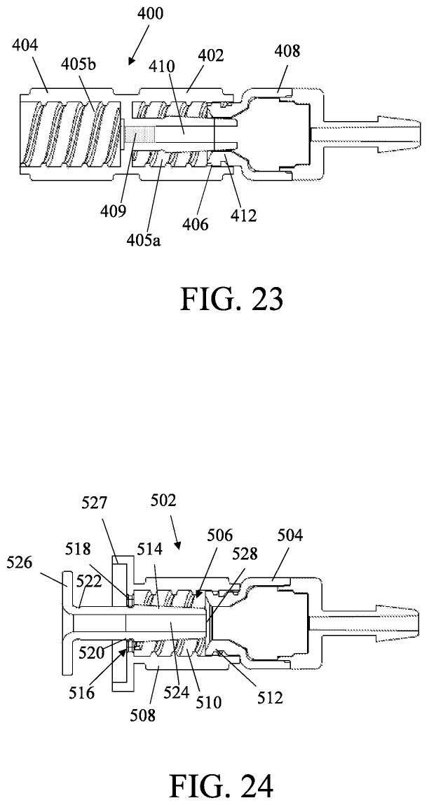

According to another non-limiting, as illustrated in FIGS. 22 and 23, the pressure release assembly 400 according to the present disclosure may be formed as a one-piece configuration. According to this embodiment, the pressure release assembly 400 may include a first threaded female side 402 having a male luer element 403 and a second threaded female side 404 without a male luer element, each having threading 405a and 405b, respectively, configured to threadably engage a threading 406 of the swabable valve 408. In certain embodiments, the non-insertion end of the male luer element may include a filter element 409 that is pervious to air but impervious to liquid, as described herein. The fluid path set including the swabable valve 408 and the one-piece pressure release assembly 400 may be shipped in the non-insertion position where the swabable valve 408 is threadably engaged with the second threaded female side 404 of the pressure release assembly 400. A lumen or throughbore 410 may be formed through the pressure release assembly 400 though the male luer element 403 of the first threaded female side 402 to the second female side 404. In this fashion, the pressure release assembly 400 may act as a dust cap, preventing contamination of the swabable valve 408. In certain embodiments (not shown) the proximal end of the male luer element 403 may be slightly recessed in the first female threaded side 402 to prevent inadvertent contact with and contamination of the proximal end of the male luer element 403. To prepare the system for a purging/priming operation, second female threaded side 404 of the one-piece pressure release assembly 400 is unthreaded from the swabable valve 408, turned 180 degrees, and rethreaded onto the swabable valve 408 such that the male luer element of the first female threaded side 402 of the pressure release assembly 400 engages and pierces the seal 412 of the swabable valve 408, allowing the purging of any air in the tubing set during the purging operation. After the purging operation, one-piece pressure release assembly 400 may be removed, the swabable valve 408 may be swabbed with a disinfected wipe and a single patient fluid path element or spike for a bulk fluid container may be attached as described herein, Alternatively, the one-piece pressure release assembly 400 may be removed again, inverted, and reattached to the swabable valve 408 by the second female side 404 so that the pressure release assembly 400, again, acts as a dust cap. The one-piece pressure release assembly 400 may be configured such that as the first female threaded side 402 is threadably screwed onto the swabable valve 408, the tip of the male luer element 403 pierces and unseals the silicone seal 412 and allows fluid communication between the atmosphere and the inner lumen of the tubing set.

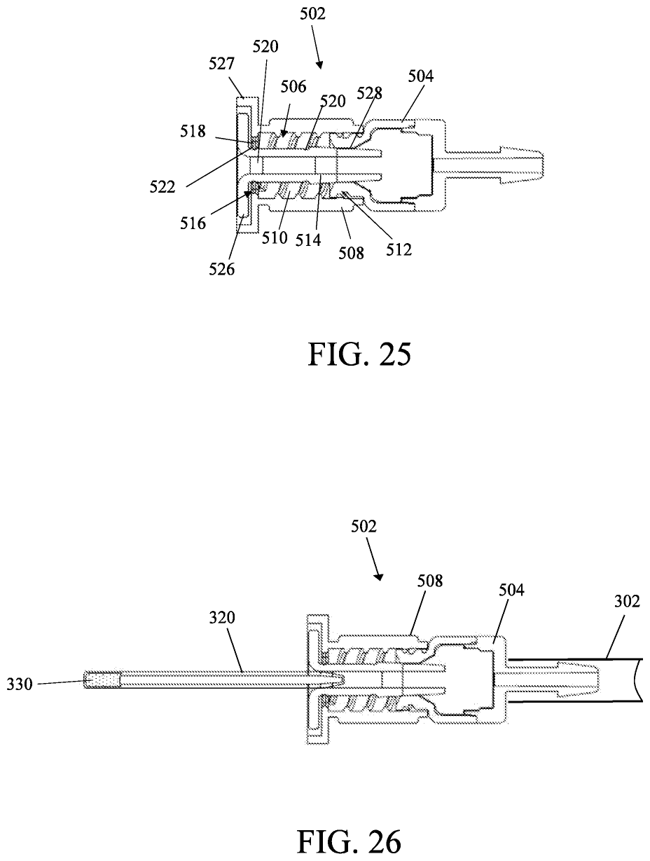

According to one non-limiting embodiment or aspect of the present disclosure, as illustrated in FIGS. 24 and 25, a pressure release assembly 502 is shown and described in detail. The pressure release assembly 502 may be substantially similar in operation as the pressure release assembly 202, with minor differences as described herein. The pressure release assembly 502 may be reversibly threadably attachable to a swabable valve 504. In one example of the present disclosure, the pressure release assembly 502 may include a connection arrangement 506 provided on an inner surface of an engagement sleeve 508 of the pressure release assembly 502. In one example, the engagement sleeve 508 may have a generally cylindrical shape. A cavity 510 may be defined by the engagement sleeve 508. In one non-limiting embodiment or aspect of the present disclosure, the connection arrangement 506 may include threading on the inner surface of the engagement sleeve 508 configured to engage threading 512 on the swabable valve 504 to secure the pressure release assembly 502 to the swabable valve 504. It is to be understood the threading of the engagement sleeve 508 and the threading 512 of the swabable valve 504 may be corresponding male and female threadings. In one example, a distal portion of the swabable valve 504 may be received in a proximal end of the engagement sleeve 508. In this example, the threading 512 of the swabable valve 504 is a male threading that is received in female threading of the connection arrangement 506 of the engagement sleeve 508.

In one non-limiting embodiment or aspect of the present disclosure, the pressure release assembly 502 of the present disclosure may include a pin member 514 that is slidably moveable from a first non-insertion position to a second, insertion position relative to the engagement sleeve 508. The engagement sleeve 508 may define an opening 516 on a distal surface thereof. The pin member 514 may be received in the opening 516 to slidably move relative to the engagement sleeve 508. One or more flexible engagement elements 518 may extend radially inward from the opening 516 to flexibly engage a detent 520, 522 defined on the slidable pin member 514. The pin member 514 may include a throughbore or lumen 524 to provide fluid communication between the proximal and distal ends of the pin member 514. In one example, the pin member 514 may include at least one vent similar to vent 221a and 221b shown in pressure release assembly 202. The distal end of the pin member 514 may include a flange 526 configured for pressing engagement, for example by manually pressing the pin member 514 to move from the first, non-insertion position to the second, insertion position. The engagement sleeve 508 may also include a flange 527 provided on a distal end of the engagement sleeve 508 that is configured to receive the flange 526 of the pin member 514 when the pin member 514 is moved to the second, insertion position.

The pin member 514 may further include a first circumferential detent 520 and a second circumferential detent 522 configured for interacting with the one or more flexible engagement elements 518 to retain the pin member 514 in the first position and the second position, respectively. The first circumferential detent 520 may be located around an approximately central portion of the pin member 514 and the second circumferential detent 522 may be located adjacent the proximal surface of the flange 526. In one example, the pin member 514 may be pushed in a first direction to advance the pin member 514 relative to the engagement sleeve 508 in a proximal direction (towards the swabable valve 504) and may be pulled in a second direction (opposite to the first direction) to advance the pin member 514 relative to the engagement sleeve 508 in a distal direction (away from the swabable valve 504). The first, non-insertion position may be understood to be a position in which the pin member 514 does not crack or open a seal 528 of the swabable valve 504, and the second, insertion position may be understood to be a position in which the pin member 514 has cracked and is inserted into the swabable valve 504 through the seal 528. In another embodiment, once the pin member 514 is pushed to the second, insertion position, the pin member 514 cannot be moved back to the first, non-insertion position. In this embodiment, the engagement sleeve 508 must be removed from the swabable valve 504 by rotating the engagement sleeve 508 relative to the swabable valve 504 to separate the pressure release assembly 502 from the swabable valve 504.

In use, the pressure release assembly 502 may be shipped in sterile packaging in the first, non-insertion position, in which the pin member 514 is in the distal position separated from the swabable valve 504. In this example of the pressure release assembly 502, the one or more flexible engagement elements 518 may be snapped into or positioned in the first detent 520 on the pin member 514 at the first, non-insertion position. In certain embodiments, the pressure release assembly 502 may be shipped with the associated swabable valve 504 and the entire pressure release assembly 502 and swabable valve 504 may be attached to the tubing and attached to the fluid injector fluid path. In other embodiments, the swabable valve 504 may be previously attached to the fluid injector fluid path, the swabable valve 504 may be disinfected, for example by swabbing with a disinfecting cloth, as described herein, and the pressure release assembly 502 may be removed from sterile packaging and threadably installed onto the swabable valve 504.

In one example of the present disclosure, the throughbore 524 of the pin member 514 may allow air from the inner lumen of the tubing connected to the swabable valve 504 to be released through the swabable valve 504 and the throughbore 524 of the pin member 514 when the pin member 514 is moved from the first, non-insertion position to the second, insertion position and the fluid injector is instructed to perform a purging or priming operation where the fluid inside the tubing is pressurized, for example by moving a piston and plunger of a syringe in the distal direction. To activate the pressure release assembly 502, the user may press the distal surface of the flange 526 to slide the pin member 514 in the proximal direction relative to the engagement sleeve 508. Upon application of pressure, the one or more flexible engagement elements 518 of the engagement sleeve 508 release from the first circumferential detent 520 and the pin member 514 slides proximally relative to the engagement sleeve 508, piercing the seal 528 of the swabable valve 504 and providing fluid communication between the throughbore or lumen 524 of the pin member 514 and the lumen of the fluid path attached to the swabable valve 504. The pin member 514 continues to slide proximally relative to the engagement sleeve 508 until the one or more flexible engagement elements 518 of the engagement sleeve 508 engage the second circumferential detent 522, holding the pressure release assembly 502 in the second, insertion position. In certain embodiments, the pressure release assembly 502 may be returned to the first position by pulling the flange 526 in the distal direction relative to the engagement sleeve 508. Alternatively, rotational removal of the engagement sleeve 508 from the swabable valve 504 removes the pin member 514 from the seal 528 of the swabable valve 504, thereby re-sealing the swabable valve 504. It is further contemplated that, once the pressure release assembly 502 has been pushed to the second, insertion position, the flange 526 cannot be pulled back in the distal direction, instead the flange 526 is locked in the flange 527 of the engagement sleeve 508, thereby preventing re-use of the pressure release assembly 502.

According to one non-limiting embodiment of the present disclosure, as illustrated in FIG. 26, it is also contemplated that a prime tube 320 may be used in conjunction with any release assembly 502 that include a full throughbore 524 defined in the pin member 514. The prime tube 320 may be inserted into a distal end of the pin member 514 such that the prime tube 320 is held in a friction fit within the throughbore 524. The prime tube 320 may include a filter material 330. Air expelled from the swabable valve 504 through the throughbore 524 may be directed into the prime tube 320.

It is worthy to note that any reference to “one embodiment” or “an embodiment” means that a particular feature, structure, or characteristic described in connection with the embodiment is included in at least one embodiment. Thus, appearances of the phrases “in one embodiment” or “in an embodiment” in various places throughout the specification are not necessarily all referring to the same embodiment. Furthermore, the particular features, structures, or characteristics may be combined in any suitable manner in one or more embodiments.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken as limiting.

With respect to the use of any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, other different components. It is to be understood that such depicted architectures are merely exemplary, and that, in fact, many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include, but are not limited to, physically mateable and/or physically interacting components, and/or wirelessly interactable, and/or wirelessly interacting components, logically interacting, and/or logically interactable components.

Some aspects may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but yet still co-operate or interact with each other.

In some instances, one or more components may be referred to herein as “configured to,” “operative,” “adapted,” etc. Those skilled in the art will recognize that “configured to” can generally encompass active-state components, and/or inactive-state components, and/or standby-state components, unless context requires otherwise.

While particular aspects of the subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

In summary, numerous benefits have been described which result from employing the concepts described herein. The foregoing disclosure has been presented for purposes of illustration and description. It is not intended to be exhaustive or limiting to the precise form disclosed. Modifications or variations are possible in light of the above teachings. It is intended that the claims submitted herewith define the overall scope of the present disclosure.

1. A pressure release assembly for a tubing set, the pressure release assembly comprising:
an engagement sleeve comprising an inner surface defining a cavity, the inner surface comprising a connection arrangement configured to connect the engagement sleeve to the tubing set and/or an associated swabable valve; and
a pin member configured to be received in the cavity of the engagement sleeve, wherein the pin member is movable between a first position in which the pin member is removed from a seal of the tubing set and/or the associated swabable valve and a second position in which the pin member is inserted into the seal of the tubing set and/or the associated swabable valve to allow a volume of air to be purged from the tubing set trough the pressure release assembly.

2. The pressure release assembly of claim 1, wherein the connection arrangement of the engagement sleeve comprises a threaded surface to threadedly connect the engagement sleeve to the tubing set.

3. The pressure release assembly of claim 1, wherein the pin member comprises a flange provided on a distal end of the pin member.

4. The pressure release assembly of claim 1, wherein the pin member defines a throughbore that extends through the pin member, and
wherein, when the pin member is held in the second position, the throughbore establishes a fluid communication with the interior of the tubing set to allow the air to be purged from the tubing set.

5. The pressure release assembly of claim 1, wherein the pin member defines at least one detent, and
wherein, when the pin member is held in the first position, a portion of the engagement sleeve engages with the at least one detent to hold the pin member in the first position.

6. (canceled)

7. The pressure release assembly of claim 1, wherein the pin member defines a first detent and a second detent, defined at a distance from the first detent, and
wherein, when the pin member is held in the first position, a portion of the engagement sleeve engages with the first detent to hold the pin member in the first position, and
wherein, when the pin member is held in the second position, the portion of the engagement sleeve engages with the second detent to hold the pin member in the second position.

8. The pressure release assembly of claim 1, wherein the pin member defines a throughbore that extends through at least a portion of the pin member,
further comprising a filter positioned in the throughbore of the pin member to permit air to move through the throughbore while preventing fluid from moving through the throughbore past the filter.

9. The pressure release assembly of any one of claims 1 to 8 claim 1, wherein an outer surface of the engagement sleeve comprises at least one gripping element.

10. The pressure release assembly of any one of claims 1 to 9 claim 1, wherein an outer surface of the pin member comprises a corresponding connection arrangement that is configured to engage with the connection arrangement of the engagement sleeve to move the pin member between the first position and the second position.

11. The pressure release assembly of any one of claims 1 to 10

1. wherein the engagement sleeve comprises a flexible arm member configured to engage at least one detent defined on the pin member.

12. (canceled)

13. The pressure release assembly of claim 1, wherein the engagement sleeve defines a vent configured to permit air to pass therethrough once the pin member has been moved to the second position.

14. A fluid injector, comprising:
a housing;
at least one syringe removably engaged with the housing;
a tubing set configured to fluidly connect the at least one syringe at a proximal end and having a swabable valve at a distal end, and at least one fluid reservoir that holds fluid to fill the at least one syringe; and
a pressure release assembly configured to connect to the distal end of the tubing set to purge air from the tubing set, the pressure release assembly comprising:
an engagement sleeve comprising an inner surface defining a cavity, the inner surface comprising a connection arrangement configured to removably connect the engagement sleeve to the tubing set; and
a pin member configured to be received in the cavity of the engagement sleeve, wherein the pin member is movable between a first position in which the pin member is removed from a seal of the swabable valve at the distal end of the tubing set and a second position in which the pin member is inserted into the seal of the swabable valve at the distal end of the tubing set to allow air to be purged from the tubing set.

15. The fluid injector of claim 14, further comprising a cassette for holding the tubing set, the cassette comprising:
a body defining at least one feature for holding at least a portion of the tubing set; and
a connecting member for removably connecting the body of the cassette to the fluid injector, wherein the cassette is configured to operatively engage the pressure release assembly to move the pressure release assembly from the first position to the second position.

16. (canceled)

17. (canceled)

18. The fluid injector of claim 14, wherein the pin member defines a throughbore that extends longitudinally through the pin member from a proximal end to a distal end, and
wherein, when the pin member is held in the second position, the throughbore establishes a fluid communication with the tubing set through an opening at the distal end of the throughbore to allow the air to be purged from the tubing set.

19. (canceled)

20. (canceled)

21. The fluid injector of claim 14, wherein the pin member defines a first detent and a second detent, defined at a distance from the first detent, and
wherein, when the pin member is held in the first position, a portion of the engagement sleeve engages with the first detent to hold the pin member in the first position, and
wherein, when the pin member is held in the second position, the portion of the engagement sleeve engages with the second detent to hold the pin member in the second position.

22.-26. (canceled)

27. The fluid injector of claim 15, further comprising an activation member provided on the cassette to engage the pin member of the pressure release assembly to move the pin member from the first position to the second position.

28. The fluid injector of claim 27, wherein the activation member comprises a solenoid valve or other electromechanical component configured to move the pin member of the pressure release assembly.

29. A method of purging air from a tubing set, the method comprising:
connecting a pressure release assembly to a distal end of the tubing set, the pressure release assembly comprising an engagement sleeve comprising an inner surface defining a cavity, the inner surface comprising a connection arrangement configured to connect the engagement sleeve to the tubing set, and a pin member configured to be received in the cavity of the engagement sleeve;
moving the pin member from a first position to a second position to break a seal at the distal end of the tubing set and insert a proximal end of the pin member into the tubing set; and
purging air from the tubing set through the pressure release assembly.

30. The method of claim 29, wherein the air is purged through one or more grooves on an outer surface of the pin member and through a vent defined in the engagement sleeve of the pressure release assembly.

31. The method of claim 29, wherein the pin member defines a throughbore extending from a proximal end of the pin member to the distal end of the pin member or to a vent through an outer surface of the pin member between the proximal end and the distal end of the pin member that establishes fluid communication with the tubing set when the pin member is in the second position such that the air is purged through the throughbore of the pin member.
